SEQUENCE LISTING (798 sequences)

SEQ ID NO: 1 - DNA, 10 bp, Artificial Sequence
  tag sequence for base balance
tcacattgct

SEQ ID NO: 2 - DNA, 10 bp, Artificial Sequence
  tag sequence for base balance
aatggcgctc

SEQ ID NO: 3 - DNA, 10 bp, Artificial Sequence
  tag sequence for base balance
gtctcaatga

SEQ ID NO: 4 - DNA, 10 bp, Artificial Sequence
  tag sequence for base balance
cggatgcaag

SEQ ID NO: 5 - DNA, 10 bp, Artificial Sequence
  tag sequence for base balance
tcgcttaagc

SEQ ID NO: 6 - DNA, 10 bp, Artificial Sequence
  tag sequence for base balance
cgaggcttag

SEQ ID NO: 7 - DNA, 10 bp, Artificial Sequence
  tag sequence for base balance
gtctaaggct

SEQ ID NO: 8 - DNA, 10 bp, Artificial Sequence
  tag sequence for base balance
aatacgccta

SEQ ID NO: 9 - DNA, 10 bp, Artificial Sequence
  tag sequence for base balance
aagcctattg

SEQ ID NO: 10 - DNA, 10 bp, Artificial Sequence
  tag sequence for base balance
cgctactgca

SEQ ID NO: 11 - DNA, 10 bp, Artificial Sequence
  tag sequence for base balance
tcaagagcat

SEQ ID NO: 12 - DNA, 10 bp, Artificial Sequence
  tag sequence for base balance
gttgtgcagc

SEQ ID NO: 13 - DNA, 10 bp, Artificial Sequence
  tag sequence for base balance
agacaggaat

SEQ ID NO: 14 - DNA, 10 bp, Artificial Sequence
  tag sequence for base balance
ccttgccgta

SEQ ID NO: 15 - DNA, 10 bp, Artificial Sequence
  tag sequence for base balance
gtcattacgg

SEQ ID NO: 16 - DNA, 10 bp, Artificial Sequence
  tag sequence for base balance
taggcattcc

SEQ ID NO: 17 - DNA, 10 bp, Artificial Sequence
  tag sequence for base balance
catatcatcg

SEQ ID NO: 18 - DNA, 10 bp, Artificial Sequence
  tag sequence for base balance
gcacaacaat

SEQ ID NO: 19 - DNA, 10 bp, Artificial Sequence
  tag sequence for base balance
ttgtcgtggc

SEQ ID NO: 20 - DNA, 10 bp, Artificial Sequence
  tag sequence for base balance
agcggtgcta

SEQ ID NO: 21 - DNA, 10 bp, Artificial Sequence
  tag sequence for base balance
agccagtagg

SEQ ID NO: 22 - DNA, 10 bp, Artificial Sequence
  tag sequence for base balance
gtaagtgtac

SEQ ID NO: 23 - DNA, 10 bp, Artificial Sequence
  tag sequence for base balance
tagtcacgtt

SEQ ID NO: 24 - DNA, 10 bp, Artificial Sequence
  tag sequence for base balance
cctgtcacca

SEQ ID NO: 25 - DNA, 10 bp, Artificial Sequence
  tag sequence for base balance
atcgtggatg

SEQ ID NO: 26 - DNA, 10 bp, Artificial Sequence
  tag sequence for base balance
tggagatcga

SEQ ID NO: 27 - DNA, 10 bp, Artificial Sequence
  tag sequence for base balance
cctcacagat

SEQ ID NO: 28 - DNA, 10 bp, Artificial Sequence
  tag sequence for base balance
gaatctctcc

SEQ ID NO: 29 - DNA, 10 bp, Artificial Sequence
  tag sequence for base balance
gcagactgac

SEQ ID NO: 30 - DNA, 10 bp, Artificial Sequence
  tag sequence for base balance
ctcattaacg

SEQ ID NO: 31 - DNA, 10 bp, Artificial Sequence
  tag sequence for base balance
tggtgagctt

SEQ ID NO: 32 - DNA, 10 bp, Artificial Sequence
  tag sequence for base balance
aatccgctga

SEQ ID NO: 33 - DNA, 10 bp, Artificial Sequence
  tag sequence for base balance
tcgcatcaac

SEQ ID NO: 34 - DNA, 10 bp, Artificial Sequence
  tag sequence for base balance
agaacagtga

SEQ ID NO: 35 - DNA, 10 bp, Artificial Sequence
  tag sequence for base balance
catgtctcct

SEQ ID NO: 36 - DNA, 10 bp, Artificial Sequence
  tag sequence for base balance
gtctggagtg

SEQ ID NO: 37 - DNA, 10 bp, Artificial Sequence
  tag sequence for base balance
gaggtctgtg

SEQ ID NO: 38 - DNA, 10 bp, Artificial Sequence
  tag sequence for base balance
ctatagacgt

SEQ ID NO: 39 - DNA, 10 bp, Artificial Sequence
  tag sequence for base balance
tgcagtgacc

SEQ ID NO: 40 - DNA, 10 bp, Artificial Sequence
  tag sequence for base balance
actccactaa

SEQ ID NO: 41 - DNA, 10 bp, Artificial Sequence
  tag sequence for base balance
gcgaagtagg

SEQ ID NO: 42 - DNA, 10 bp, Artificial Sequence
  tag sequence for base balance
tgcctaacct

SEQ ID NO: 43 - DNA, 10 bp, Artificial Sequence
  tag sequence for base balance
aatggtctac

SEQ ID NO: 44 - DNA, 10 bp, Artificial Sequence
  tag sequence for base balance
ctatccggta

SEQ ID NO: 45 - DNA, 10 bp, Artificial Sequence
  tag sequence for base balance
tacgcttcag

SEQ ID NO: 46 - DNA, 10 bp, Artificial Sequence
  tag sequence for base balance
cggagcatct

SEQ ID NO: 47 - DNA, 10 bp, Artificial Sequence
  tag sequence for base balance
gtactagatc

SEQ ID NO: 48 - DNA, 10 bp, Artificial Sequence
  tag sequence for base balance
acttagcgga

SEQ ID NO: 49 - DNA, 10 bp, Artificial Sequence
  tag sequence for base balance
atcactccat

SEQ ID NO: 50 - DNA, 10 bp, Artificial Sequence
  tag sequence for base balance
gatcgcagtg

SEQ ID NO: 51 - DNA, 10 bp, Artificial Sequence
  tag sequence for base balance
ccggaattcc

SEQ ID NO: 52 - DNA, 10 bp, Artificial Sequence
  tag sequence for base balance
tgattggaga

SEQ ID NO: 53 - DNA, 10 bp, Artificial Sequence
  tag sequence for base balance
cacaaggtcg

SEQ ID NO: 54 - DNA, 10 bp, Artificial Sequence
  tag sequence for base balance
tctcgcagga

SEQ ID NO: 55 - DNA, 10 bp, Artificial Sequence
  tag sequence for base balance
gtggtatcat

SEQ ID NO: 56 - DNA, 10 bp, Artificial Sequence
  tag sequence for base balance
agatctcatc

SEQ ID NO: 57 - DNA, 10 bp, Artificial Sequence
  tag sequence for base balance
gatggagatt

SEQ ID NO: 58 - DNA, 10 bp, Artificial Sequence
  tag sequence for base balance
ctcattctgc

SEQ ID NO: 59 - DNA, 10 bp, Artificial Sequence
  tag sequence for base balance
tggcagacaa

SEQ ID NO: 60 - DNA, 10 bp, Artificial Sequence
  tag sequence for base balance
acatcctgcg

SEQ ID NO: 61 - DNA, 10 bp, Artificial Sequence
  tag sequence for base balance
acgtcgcaga

SEQ ID NO: 62 - DNA, 10 bp, Artificial Sequence
  tag sequence for base balance
tgtataggct

SEQ ID NO: 63 - DNA, 10 bp, Artificial Sequence
  tag sequence for base balance
caacacattg

SEQ ID NO: 64 - DNA, 10 bp, Artificial Sequence
  tag sequence for base balance
gtcggttcac

SEQ ID NO: 65 - DNA, 10 bp, Artificial Sequence
  tag sequence for base balance
agccataagc

SEQ ID NO: 66 - DNA, 10 bp, Artificial Sequence
  tag sequence for base balance
gtattccgag

SEQ ID NO: 67 - DNA, 10 bp, Artificial Sequence
  tag sequence for base balance
cataggttca

SEQ ID NO: 68 - DNA, 10 bp, Artificial Sequence
  tag sequence for base balance
tcggcagctt

SEQ ID NO: 69 - DNA, 10 bp, Artificial Sequence
  tag sequence for base balance
gttcggtcct

SEQ ID NO: 70 - DNA, 10 bp, Artificial Sequence
  tag sequence for base balance
tggattgtag

SEQ ID NO: 71 - DNA, 10 bp, Artificial Sequence
  tag sequence for base balance
ccagaacgtc

SEQ ID NO: 72 - DNA, 10 bp, Artificial Sequence
  tag sequence for base balance
aactccaaga

SEQ ID NO: 73 - DNA, 10 bp, Artificial Sequence
  tag sequence for base balance
cgtacactgg

SEQ ID NO: 74 - DNA, 10 bp, Artificial Sequence
  tag sequence for base balance
gcacacagca

SEQ ID NO: 75 - DNA, 10 bp, Artificial Sequence
  tag sequence for base balance
atggtgtatc

SEQ ID NO: 76 - DNA, 10 bp, Artificial Sequence
  tag sequence for base balance
tactgtgcat

SEQ ID NO: 77 - DNA, 10 bp, Artificial Sequence
  tag sequence for base balance
cggcaatcag

SEQ ID NO: 78 - DNA, 10 bp, Artificial Sequence
  tag sequence for base balance
gtagttcgga

SEQ ID NO: 79 - DNA, 10 bp, Artificial Sequence
  tag sequence for base balance
tacaggaact

SEQ ID NO: 80 - DNA, 10 bp, Artificial Sequence
  tag sequence for base balance
acttccgttc

SEQ ID NO: 81 - DNA, 10 bp, Artificial Sequence
  tag sequence for base balance
tgctccacga

SEQ ID NO: 82 - DNA, 10 bp, Artificial Sequence
  tag sequence for base balance
aatcaaggtc

SEQ ID NO: 83 - DNA, 10 bp, Artificial Sequence
  tag sequence for base balance
gtaggtcaat

SEQ ID NO: 84 - DNA, 10 bp, Artificial Sequence
  tag sequence for base balance
ccgatgttcg

SEQ ID NO: 85 - DNA, 10 bp, Artificial Sequence
  tag sequence for base balance
gacgtgtgca

SEQ ID NO: 86 - DNA, 10 bp, Artificial Sequence
  tag sequence for base balance
tcgccacttc

SEQ ID NO: 87 - DNA, 10 bp, Artificial Sequence
  tag sequence for base balance
ataagcacgt

SEQ ID NO: 88 - DNA, 10 bp, Artificial Sequence
  tag sequence for base balance
cgttatgaag

SEQ ID NO: 89 - DNA, 10 bp, Artificial Sequence
  tag sequence for base balance
aatagagcca

SEQ ID NO: 90 - DNA, 10 bp, Artificial Sequence
  tag sequence for base balance
gtacctcgac

SEQ ID NO: 91 - DNA, 10 bp, Artificial Sequence
  tag sequence for base balance
ccggtgattg

SEQ ID NO: 92 - DNA, 10 bp, Artificial Sequence
  tag sequence for base balance
tgctactagt

SEQ ID NO: 93 - DNA, 10 bp, Artificial Sequence
  tag sequence for base balance
gatccggact

SEQ ID NO: 94 - DNA, 10 bp, Artificial Sequence
  tag sequence for base balance
ttaggcacaa

SEQ ID NO: 95 - DNA, 10 bp, Artificial Sequence
  tag sequence for base balance
agcattcttg

SEQ ID NO: 96 - DNA, 10 bp, Artificial Sequence
  tag sequence for base balance
ccgtaatggc

SEQ ID NO: 97 - DNA, 10 bp, Artificial Sequence
  tag sequence for base balance
gtatagctgc

SEQ ID NO: 98 - DNA, 10 bp, Artificial Sequence
  tag sequence for base balance
cagacatctg

SEQ ID NO: 99 - DNA, 10 bp, Artificial Sequence
  tag sequence for base balance
agcggtggat

SEQ ID NO: 100 - DNA, 10 bp, Artificial Sequence
  tag sequence for base balance
tctctcaaca

SEQ ID NO: 101 - DNA, 10 bp, Artificial Sequence
  tag sequence for base balance
catccactgt

SEQ ID NO: 102 - DNA, 10 bp, Artificial Sequence
  tag sequence for base balance
gccgtgaaca

SEQ ID NO: 103 - DNA, 10 bp, Artificial Sequence
  tag sequence for base balance
ataagcggag

SEQ ID NO: 104 - DNA, 10 bp, Artificial Sequence
  tag sequence for base balance
tggtattctc

SEQ ID NO: 105 - DNA, 10 bp, Artificial Sequence
  tag sequence for base balance
acagttctca

SEQ ID NO: 106 - DNA, 10 bp, Artificial Sequence
  tag sequence for base balance
cattgagagc

SEQ ID NO: 107 - DNA, 10 bp, Artificial Sequence
  tag sequence for base balance
gtcacgactg

SEQ ID NO: 108 - DNA, 10 bp, Artificial Sequence
  tag sequence for base balance
tggcactgat

SEQ ID NO: 109 - DNA, 10 bp, Artificial Sequence
  tag sequence for base balance
aatgattcgc

SEQ ID NO: 110 - DNA, 10 bp, Artificial Sequence
  tag sequence for base balance
cgcttaagta

SEQ ID NO: 111 - DNA, 10 bp, Artificial Sequence
  tag sequence for base balance
ttgagcgacg

SEQ ID NO: 112 - DNA, 10 bp, Artificial Sequence
  tag sequence for base balance
gcaccgctat

SEQ ID NO: 113 - DNA, 10 bp, Artificial Sequence
  tag sequence for base balance
acgaacggat

SEQ ID NO: 114 - DNA, 10 bp, Artificial Sequence
  tag sequence for base balance
gaacggacta

SEQ ID NO: 115 - DNA, 10 bp, Artificial Sequence
  tag sequence for base balance
tgctctctgg

SEQ ID NO: 116 - DNA, 10 bp, Artificial Sequence
  tag sequence for base balance
cttgtatacc

SEQ ID NO: 117 - DNA, 10 bp, Artificial Sequence
  tag sequence for base balance
caccagcaca

SEQ ID NO: 118 - DNA, 10 bp, Artificial Sequence
  tag sequence for base balance
ttgtctgtag

SEQ ID NO: 119 - DNA, 10 bp, Artificial Sequence
  tag sequence for base balance
agtatcactc

SEQ ID NO: 120 - DNA, 10 bp, Artificial Sequence
  tag sequence for base balance
gcaggatggt

SEQ ID NO: 121 - DNA, 10 bp, Artificial Sequence
  tag sequence for base balance
ttatccacgt

SEQ ID NO: 122 - DNA, 10 bp, Artificial Sequence
  tag sequence for base balance
acggttcgtc

SEQ ID NO: 123 - DNA, 10 bp, Artificial Sequence
  tag sequence for base balance
gaccagtaag

SEQ ID NO: 124 - DNA, 10 bp, Artificial Sequence
  tag sequence for base balance
cgtagagtca

SEQ ID NO: 125 - DNA, 10 bp, Artificial Sequence
  tag sequence for base balance
acgttaaggt

SEQ ID NO: 126 - DNA, 10 bp, Artificial Sequence
  tag sequence for base balance
ctcagcttag

SEQ ID NO: 127 - DNA, 10 bp, Artificial Sequence
  tag sequence for base balance
tgtgctccta

SEQ ID NO: 128 - DNA, 10 bp, Artificial Sequence
  tag sequence for base balance
gaacaggacc

SEQ ID NO: 129 - DNA, 10 bp, Artificial Sequence
  tag sequence for base balance
gcgaccttga

SEQ ID NO: 130 - DNA, 10 bp, Artificial Sequence
  tag sequence for base balance
atcgtgagtt

SEQ ID NO: 131 - DNA, 10 bp, Artificial Sequence
  tag sequence for base balance
cgtcgtcaac

SEQ ID NO: 132 - DNA, 10 bp, Artificial Sequence
  tag sequence for base balance
taataagccg

SEQ ID NO: 133 - DNA, 10 bp, Artificial Sequence
  tag sequence for base balance
ccacttagta

SEQ ID NO: 134 - DNA, 10 bp, Artificial Sequence
  tag sequence for base balance
agtagctagt

SEQ ID NO: 135 - DNA, 10 bp, Artificial Sequence
  tag sequence for base balance
gacgaactcg

SEQ ID NO: 136 - DNA, 10 bp, Artificial Sequence
  tag sequence for base balance
ttgtcggcac

SEQ ID NO: 137 - DNA, 10 bp, Artificial Sequence
  tag sequence for base balance
agcatatcgt

SEQ ID NO: 138 - DNA, 10 bp, Artificial Sequence
  tag sequence for base balance
gtagacggag

SEQ ID NO: 139 - DNA, 10 bp, Artificial Sequence
  tag sequence for base balance
cattctcatc

SEQ ID NO: 140 - DNA, 10 bp, Artificial Sequence
  tag sequence for base balance
tcgcggatca

SEQ ID NO: 141 - DNA, 10 bp, Artificial Sequence
  tag sequence for base balance
ctggaggcaa

SEQ ID NO: 142 - DNA, 10 bp, Artificial Sequence
  tag sequence for base balance
gatacttgtg

SEQ ID NO: 143 - DNA, 10 bp, Artificial Sequence
  tag sequence for base balance
tgcctccact

SEQ ID NO: 144 - DNA, 10 bp, Artificial Sequence
  tag sequence for base balance
acatgaatgc

SEQ ID NO: 145 - DNA, 10 bp, Artificial Sequence
  tag sequence for base balance
tcagcagagg

SEQ ID NO: 146 - DNA, 10 bp, Artificial Sequence
  tag sequence for base balance
ctgtgcatta

SEQ ID NO: 147 - DNA, 10 bp, Artificial Sequence
  tag sequence for base balance
agtaatcgac

SEQ ID NO: 148 - DNA, 10 bp, Artificial Sequence
  tag sequence for base balance
agtaatcgac

SEQ ID NO: 149 - DNA, 10 bp, Artificial Sequence
  tag sequence for base balance
aagcggtgaa

SEQ ID NO: 150 - DNA, 10 bp, Artificial Sequence
  tag sequence for base balance
ctatcacact

SEQ ID NO: 151 - DNA, 10 bp, Artificial Sequence
  tag sequence for base balance
gccgttatgc

SEQ ID NO: 152 - DNA, 10 bp, Artificial Sequence
  tag sequence for base balance
tgtaacgctg

SEQ ID NO: 153 - DNA, 10 bp, Artificial Sequence
  tag sequence for base balance
gcgtgtaact

SEQ ID NO: 154 - DNA, 10 bp, Artificial Sequence
  tag sequence for base balance
catgtaccac

SEQ ID NO: 155 - DNA, 10 bp, Artificial Sequence
  tag sequence for base balance
tgccactgta

SEQ ID NO: 156 - DNA, 10 bp, Artificial Sequence
  tag sequence for base balance
ataacggtgg

SEQ ID NO: 157 - DNA, 10 bp, Artificial Sequence
  tag sequence for base balance
cttgaaggtt

SEQ ID NO: 158 - DNA, 10 bp, Artificial Sequence
  tag sequence for base balance
gcctctatgg

SEQ ID NO: 159 - DNA, 10 bp, Artificial Sequence
  tag sequence for base balance
tagatccacc

SEQ ID NO: 160 - DNA, 10 bp, Artificial Sequence
  tag sequence for base balance
agacggtcaa

SEQ ID NO: 161 - DNA, 10 bp, Artificial Sequence
  tag sequence for base balance
gctggattaa

SEQ ID NO: 162 - DNA, 10 bp, Artificial Sequence
  tag sequence for base balance
taacaccggc

SEQ ID NO: 163 - DNA, 10 bp, Artificial Sequence
  tag sequence for base balance
atgactgccg

SEQ ID NO: 164 - DNA, 10 bp, Artificial Sequence
  tag sequence for base balance
cgcttgaatt

SEQ ID NO: 165 - DNA, 10 bp, Artificial Sequence
  tag sequence for base balance
ctatagcgag

SEQ ID NO: 166 - DNA, 10 bp, Artificial Sequence
  tag sequence for base balance
aacgttgttc

SEQ ID NO: 167 - DNA, 10 bp, Artificial Sequence
  tag sequence for base balance
gctaccacgt

SEQ ID NO: 168 - DNA, 10 bp, Artificial Sequence
  tag sequence for base balance
tggcgataca

SEQ ID NO: 169 - DNA, 10 bp, Artificial Sequence
  tag sequence for base balance
tccgccaatc

SEQ ID NO: 170 - DNA, 10 bp, Artificial Sequence
  tag sequence for base balance
caactagtgt

SEQ ID NO: 171 - DNA, 10 bp, Artificial Sequence
  tag sequence for base balance
agtaagccaa

SEQ ID NO: 172 - DNA, 10 bp, Artificial Sequence
  tag sequence for base balance
gtgtgttgcg

SEQ ID NO: 173 - DNA, 10 bp, Artificial Sequence
  tag sequence for base balance
gatcagatgg

SEQ ID NO: 174 - DNA, 10 bp, Artificial Sequence
  tag sequence for base balance
ctcgtaggtc

SEQ ID NO: 175 - DNA, 10 bp, Artificial Sequence
  tag sequence for base balance
tcgtgtccat

SEQ ID NO: 176 - DNA, 10 bp, Artificial Sequence
  tag sequence for base balance
agaacctaca

SEQ ID NO: 177 - DNA, 10 bp, Artificial Sequence
  tag sequence for base balance
ccgcattcct

SEQ ID NO: 178 - DNA, 10 bp, Artificial Sequence
  tag sequence for base balance
gttggacata

SEQ ID NO: 179 - DNA, 10 bp, Artificial Sequence
  tag sequence for base balance
tgatcgaggc

SEQ ID NO: 180 - DNA, 10 bp, Artificial Sequence
  tag sequence for base balance
aacatcgtag

SEQ ID NO: 181 - DNA, 10 bp, Artificial Sequence
  tag sequence for base balance
gttgcgcgaa

SEQ ID NO: 182 - DNA, 10 bp, Artificial Sequence
  tag sequence for base balance
acaagtaagc

SEQ ID NO: 183 - DNA, 10 bp, Artificial Sequence
  tag sequence for base balance
cgcttctcct

SEQ ID NO: 184 - DNA, 10 bp, Artificial Sequence
  tag sequence for base balance
tagcaagttg

SEQ ID NO: 185 - DNA, 10 bp, Artificial Sequence
  tag sequence for base balance
aggcctcttc

SEQ ID NO: 186 - DNA, 10 bp, Artificial Sequence
  tag sequence for base balance
gtaatgtcgt

SEQ ID NO: 187 - DNA, 10 bp, Artificial Sequence
  tag sequence for base balance
cactgcagag

SEQ ID NO: 188 - DNA, 10 bp, Artificial Sequence
  tag sequence for base balance
tctgaagaca

SEQ ID NO: 189 - DNA, 10 bp, Artificial Sequence
  tag sequence for base balance
gctaaggata

SEQ ID NO: 190 - DNA, 10 bp, Artificial Sequence
  tag sequence for base balance
cacggttggt

SEQ ID NO: 191 - DNA, 10 bp, Artificial Sequence
  tag sequence for base balance
tgaccaccag

SEQ ID NO: 192 - DNA, 10 bp, Artificial Sequence
  tag sequence for base balance
atgttcatcc

SEQ ID NO: 193 - DNA, 10 bp, Artificial Sequence
  tag sequence for base balance
agctactctg

SEQ ID NO: 194 - DNA, 10 bp, Artificial Sequence
  tag sequence for base balance
caacgtgagt

SEQ ID NO: 195 - DNA, 10 bp, Artificial Sequence
  tag sequence for base balance
tcgatgctaa

SEQ ID NO: 196 - DNA, 10 bp, Artificial Sequence
  tag sequence for base balance
gttgcaagcc

SEQ ID NO: 197 - DNA, 10 bp, Artificial Sequence
  tag sequence for base balance
cgacatgtgt

SEQ ID NO: 198 - DNA, 10 bp, Artificial Sequence
  tag sequence for base balance
gatgcgcata

SEQ ID NO: 199 - DNA, 10 bp, Artificial Sequence
  tag sequence for base balance
tcgtgatcag

SEQ ID NO: 200 - DNA, 10 bp, Artificial Sequence
  tag sequence for base balance
atcatcagcc

SEQ ID NO: 201 - DNA, 10 bp, Artificial Sequence
  tag sequence for base balance
tcaatggcgg

SEQ ID NO: 202 - DNA, 10 bp, Artificial Sequence
  tag sequence for base balance
cagtaactct

SEQ ID NO: 203 - DNA, 10 bp, Artificial Sequence
  tag sequence for base balance
gttgcctgac

SEQ ID NO: 204 - DNA, 10 bp, Artificial Sequence
  tag sequence for base balance
agccgtaata

SEQ ID NO: 205 - DNA, 10 bp, Artificial Sequence
  tag sequence for base balance
ctaataggct

SEQ ID NO: 206 - DNA, 10 bp, Artificial Sequence
  tag sequence for base balance
tctcctccac

SEQ ID NO: 207 - DNA, 10 bp, Artificial Sequence
  tag sequence for base balance
agctgcatga

SEQ ID NO: 208 - DNA, 10 bp, Artificial Sequence
  tag sequence for base balance
gaggagtatg

SEQ ID NO: 209 - DNA, 10 bp, Artificial Sequence
  tag sequence for base balance
accacgtagc

SEQ ID NO: 210 - DNA, 10 bp, Artificial Sequence
  tag sequence for base balance
gaatgcagta

SEQ ID NO: 211 - DNA, 10 bp, Artificial Sequence
  tag sequence for base balance
ttggtagcct

SEQ ID NO: 212 - DNA, 10 bp, Artificial Sequence
  tag sequence for base balance
cgtcatctag

SEQ ID NO: 213 - DNA, 10 bp, Artificial Sequence
  tag sequence for base balance
tcaatgaggt

SEQ ID NO: 214 - DNA, 10 bp, Artificial Sequence
  tag sequence for base balance
agcgaagctg

SEQ ID NO: 215 - DNA, 10 bp, Artificial Sequence
  tag sequence for base balance
ctgtcttaac

SEQ ID NO: 216 - DNA, 10 bp, Artificial Sequence
  tag sequence for base balance
gatcgcctca

SEQ ID NO: 217 - DNA, 10 bp, Artificial Sequence
  tag sequence for base balance
gttccgaatg

SEQ ID NO: 218 - DNA, 10 bp, Artificial Sequence
  tag sequence for base balance
tacgtacgca

SEQ ID NO: 219 - DNA, 10 bp, Artificial Sequence
  tag sequence for base balance
cggagttcac

SEQ ID NO: 220 - DNA, 10 bp, Artificial Sequence
  tag sequence for base balance
acatacgtgt

SEQ ID NO: 221 - DNA, 10 bp, Artificial Sequence
  tag sequence for base balance
ctgaagagat

SEQ ID NO: 222 - DNA, 10 bp, Artificial Sequence
  tag sequence for base balance
gaagcctcca

SEQ ID NO: 223 - DNA, 10 bp, Artificial Sequence
  tag sequence for base balance
tgtcttcatc

SEQ ID NO: 224 - DNA, 10 bp, Artificial Sequence
  tag sequence for base balance
acctgagtgg

SEQ ID NO: 225 - DNA, 10 bp, Artificial Sequence
  tag sequence for base balance
gtacgtcctt

SEQ ID NO: 226 - DNA, 10 bp, Artificial Sequence
  tag sequence for base balance
aactaggtca

SEQ ID NO: 227 - DNA, 10 bp, Artificial Sequence
  tag sequence for base balance
tgtgtcaggc

SEQ ID NO: 228 - DNA, 10 bp, Artificial Sequence
  tag sequence for base balance
ccgacataag

SEQ ID NO: 229 - DNA, 10 bp, Artificial Sequence
  tag sequence for base balance
tccacacgtc

SEQ ID NO: 230 - DNA, 10 bp, Artificial Sequence
  tag sequence for base balance
cggcacatga

SEQ ID NO: 231 - DNA, 10 bp, Artificial Sequence
  tag sequence for base balance
gtatgttcct

SEQ ID NO: 232 - DNA, 10 bp, Artificial Sequence
  tag sequence for base balance
aatgtggaag

SEQ ID NO: 233 - DNA, 10 bp, Artificial Sequence
  tag sequence for base balance
agacagacgt

SEQ ID NO: 234 - DNA, 10 bp, Artificial Sequence
  tag sequence for base balance
ttctgtggag

SEQ ID NO: 235 - DNA, 10 bp, Artificial Sequence
  tag sequence for base balance
caggtcttcc

SEQ ID NO: 236 - DNA, 10 bp, Artificial Sequence
  tag sequence for base balance
gctacacata

SEQ ID NO: 237 - DNA, 10 bp, Artificial Sequence
  tag sequence for base balance
ctagcgacac

SEQ ID NO: 238 - DNA, 10 bp, Artificial Sequence
  tag sequence for base balance
aggcattact

SEQ ID NO: 239 - DNA, 10 bp, Artificial Sequence
  tag sequence for base balance
gacatccgga

SEQ ID NO: 240 - DNA, 10 bp, Artificial Sequence
  tag sequence for base balance
tcttgagttg

SEQ ID NO: 241 - DNA, 10 bp, Artificial Sequence
  tag sequence for base balance
acaacagaag

SEQ ID NO: 242 - DNA, 10 bp, Artificial Sequence
  tag sequence for base balance
cgcttgtgga

SEQ ID NO: 243 - DNA, 10 bp, Artificial Sequence
  tag sequence for base balance
gttggtctcc

SEQ ID NO: 244 - DNA, 10 bp, Artificial Sequence
  tag sequence for base balance
tagcacactt

SEQ ID NO: 245 - DNA, 10 bp, Artificial Sequence
  tag sequence for base balance
gcttgcaata

SEQ ID NO: 246 - DNA, 10 bp, Artificial Sequence
  tag sequence for base balance
tggacgtgct

SEQ ID NO: 247 - DNA, 10 bp, Artificial Sequence
  tag sequence for base balance
aacgaaccgg

SEQ ID NO: 248 - DNA, 10 bp, Artificial Sequence
  tag sequence for base balance
ctacttgtac

SEQ ID NO: 249 - DNA, 10 bp, Artificial Sequence
  tag sequence for base balance
cggtgagtga

SEQ ID NO: 250 - DNA, 10 bp, Artificial Sequence
  tag sequence for base balance
gcaatgcatt

SEQ ID NO: 251 - DNA, 10 bp, Artificial Sequence
  tag sequence for base balance
ttccattcac

SEQ ID NO: 252 - DNA, 10 bp, Artificial Sequence
  tag sequence for base balance
aatgccagcg

SEQ ID NO: 253 - DNA, 10 bp, Artificial Sequence
  tag sequence for base balance
tactcttctc

SEQ ID NO: 254 - DNA, 10 bp, Artificial Sequence
  tag sequence for base balance
aggaaggtaa

SEQ ID NO: 255 - DNA, 10 bp, Artificial Sequence
  tag sequence for base balance
gttggacggt

SEQ ID NO: 256 - DNA, 10 bp, Artificial Sequence
  tag sequence for base balance
ccactcaacg

SEQ ID NO: 257 - DNA, 10 bp, Artificial Sequence
  tag sequence for base balance
aatgactggt

SEQ ID NO: 258 - DNA, 10 bp, Artificial Sequence
  tag sequence for base balance
gtgccacaac

SEQ ID NO: 259 - DNA, 10 bp, Artificial Sequence
  tag sequence for base balance
cgaatgatcg

SEQ ID NO: 260 - DNA, 10 bp, Artificial Sequence
  tag sequence for base balance
tcctgtgcta

SEQ ID NO: 261 - DNA, 10 bp, Artificial Sequence
  tag sequence for base balance
tgtgaattgg

SEQ ID NO: 262 - DNA, 10 bp, Artificial Sequence
  tag sequence for base balance
aaccggcctt

SEQ ID NO: 263 - DNA, 10 bp, Artificial Sequence
  tag sequence for base balance
ctatccgacc

SEQ ID NO: 264 - DNA, 10 bp, Artificial Sequence
  tag sequence for base balance
gcgattagaa

SEQ ID NO: 265 - DNA, 10 bp, Artificial Sequence
  tag sequence for base balance
cgtaaccgca

SEQ ID NO: 266 - DNA, 10 bp, Artificial Sequence
  tag sequence for base balance
gactgataac

SEQ ID NO: 267 - DNA, 10 bp, Artificial Sequence
  tag sequence for base balance
atgcttactg

SEQ ID NO: 268 - DNA, 10 bp, Artificial Sequence
  tag sequence for base balance
tcagcggtgt

SEQ ID NO: 269 - DNA, 10 bp, Artificial Sequence
  tag sequence for base balance
acataacacc

SEQ ID NO: 270 - DNA, 10 bp, Artificial Sequence
  tag sequence for base balance
cacctgaggt

SEQ ID NO: 271 - DNA, 10 bp, Artificial Sequence
  tag sequence for base balance
gtgactgtaa

SEQ ID NO: 272 - DNA, 10 bp, Artificial Sequence
  tag sequence for base balance
tgtggctctg

SEQ ID NO: 273 - DNA, 10 bp, Artificial Sequence
  tag sequence for base balance
ctcagactct

SEQ ID NO: 274 - DNA, 10 bp, Artificial Sequence
  tag sequence for base balance
acacatgcta

SEQ ID NO: 275 - DNA, 10 bp, Artificial Sequence
  tag sequence for base balance
tgtgcctaag

SEQ ID NO: 276 - DNA, 10 bp, Artificial Sequence
  tag sequence for base balance
gagttgaggc

SEQ ID NO: 277 - DNA, 10 bp, Artificial Sequence
  tag sequence for base balance
agccgttctc

SEQ ID NO: 278 - DNA, 10 bp, Artificial Sequence
  tag sequence for base balance
ttgttggtct

SEQ ID NO: 279 - DNA, 10 bp, Artificial Sequence
  tag sequence for base balance
caagacaaga

SEQ ID NO: 280 - DNA, 10 bp, Artificial Sequence
  tag sequence for base balance
gctacacgag

SEQ ID NO: 281 - DNA, 10 bp, Artificial Sequence
  tag sequence for base balance
gtgacgcgat

SEQ ID NO: 282 - DNA, 10 bp, Artificial Sequence
  tag sequence for base balance
aacctctctg

SEQ ID NO: 283 - DNA, 10 bp, Artificial Sequence
  tag sequence for base balance
tcttgagaga

SEQ ID NO: 284 - DNA, 10 bp, Artificial Sequence
  tag sequence for base balance
cgagatatcc

SEQ ID NO: 285 - DNA, 10 bp, Artificial Sequence
  tag sequence for base balance
gaaggattca

SEQ ID NO: 286 - DNA, 10 bp, Artificial Sequence
  tag sequence for base balance
tcgcctggtt

SEQ ID NO: 287 - DNA, 10 bp, Artificial Sequence
  tag sequence for base balance
agtaacacgg

SEQ ID NO: 288 - DNA, 10 bp, Artificial Sequence
  tag sequence for base balance
ctcttgcaac

SEQ ID NO: 289 - DNA, 10 bp, Artificial Sequence
  tag sequence for base balance
agagacttac

SEQ ID NO: 290 - DNA, 10 bp, Artificial Sequence
  tag sequence for base balance
cttatggccg

SEQ ID NO: 291 - DNA, 10 bp, Artificial Sequence
  tag sequence for base balance
gcctgacgtt

SEQ ID NO: 292 - DNA, 10 bp, Artificial Sequence
  tag sequence for base balance
tagcctaaga

SEQ ID NO: 293 - DNA, 10 bp, Artificial Sequence
  tag sequence for base balance
aagcatatcc

SEQ ID NO: 294 - DNA, 10 bp, Artificial Sequence
  tag sequence for base balance
ctaatccgtt

SEQ ID NO: 295 - DNA, 10 bp, Artificial Sequence
  tag sequence for base balance
gcttggtcga

SEQ ID NO: 296 - DNA, 10 bp, Artificial Sequence
  tag sequence for base balance
tgcgcagaag

SEQ ID NO: 297 - DNA, 10 bp, Artificial Sequence
  tag sequence for base balance
gatcctgata

SEQ ID NO: 298 - DNA, 10 bp, Artificial Sequence
  tag sequence for base balance
tcaagcacgg

SEQ ID NO: 299 - DNA, 10 bp, Artificial Sequence
  tag sequence for base balance
ctgtagcgct

SEQ ID NO: 300 - DNA, 10 bp, Artificial Sequence
  tag sequence for base balance
agcgtattac

SEQ ID NO: 301 - DNA, 10 bp, Artificial Sequence
  tag sequence for base balance
tgtctgattg

SEQ ID NO: 302 - DNA, 10 bp, Artificial Sequence
  tag sequence for base balance
accagacggt

SEQ ID NO: 303 - DNA, 10 bp, Artificial Sequence
  tag sequence for base balance
gtgtctgacc

SEQ ID NO: 304 - DNA, 10 bp, Artificial Sequence
  tag sequence for base balance
caagactcaa

SEQ ID NO: 305 - DNA, 10 bp, Artificial Sequence
  tag sequence for base balance
caagactcaa

SEQ ID NO: 306 - DNA, 10 bp, Artificial Sequence
  tag sequence for base balance
agacgaggtc

SEQ ID NO: 307 - DNA, 10 bp, Artificial Sequence
  tag sequence for base balance
ctggattact

SEQ ID NO: 308 - DNA, 10 bp, Artificial Sequence
  tag sequence for base balance
gatatgctga

SEQ ID NO: 309 - DNA, 10 bp, Artificial Sequence
  tag sequence for base balance
ctggtcaagg

SEQ ID NO: 310 - DNA, 10 bp, Artificial Sequence
  tag sequence for base balance
gctccattcc

SEQ ID NO: 311 - DNA, 10 bp, Artificial Sequence
  tag sequence for base balance
tgatgtcgaa

SEQ ID NO: 312 - DNA, 10 bp, Artificial Sequence
  tag sequence for base balance
aacaaggctt

SEQ ID NO: 313 - DNA, 10 bp, Artificial Sequence
  tag sequence for base balance
catctagaca

SEQ ID NO: 314 - DNA, 10 bp, Artificial Sequence
  tag sequence for base balance
gcggatacag

SEQ ID NO: 315 - DNA, 10 bp, Artificial Sequence
  tag sequence for base balance
ttctgcttgt

SEQ ID NO: 316 - DNA, 10 bp, Artificial Sequence
  tag sequence for base balance
agaacgcgtc

SEQ ID NO: 317 - DNA, 10 bp, Artificial Sequence
  tag sequence for base balance
aagacgaact

SEQ ID NO: 318 - DNA, 10 bp, Artificial Sequence
  tag sequence for base balance
ccagtactgc

SEQ ID NO: 319 - DNA, 10 bp, Artificial Sequence
  tag sequence for base balance
gttcgctgta

SEQ ID NO: 320 - DNA, 10 bp, Artificial Sequence
  tag sequence for base balance
tgctatgcag

SEQ ID NO: 321 - DNA, 10 bp, Artificial Sequence
  tag sequence for base balance
tacacgcgca

SEQ ID NO: 322 - DNA, 10 bp, Artificial Sequence
  tag sequence for base balance
aggtacgcag

SEQ ID NO: 323 - DNA, 10 bp, Artificial Sequence
  tag sequence for base balance
gttcgattgt

SEQ ID NO: 324 - DNA, 10 bp, Artificial Sequence
  tag sequence for base balance
ccagttaatc

SEQ ID NO: 325 - DNA, 10 bp, Artificial Sequence
  tag sequence for base balance
taagatcgga

SEQ ID NO: 326 - DNA, 10 bp, Artificial Sequence
  tag sequence for base balance
agctcggctt

SEQ ID NO: 327 - DNA, 10 bp, Artificial Sequence
  tag sequence for base balance
gttcgataag

SEQ ID NO: 328 - DNA, 10 bp, Artificial Sequence
  tag sequence for base balance
ccgatcatcc

SEQ ID NO: 329 - DNA, 10 bp, Artificial Sequence
  tag sequence for base balance
actggactca

SEQ ID NO: 330 - DNA, 10 bp, Artificial Sequence
  tag sequence for base balance
caatagaggc

SEQ ID NO: 331 - DNA, 10 bp, Artificial Sequence
  tag sequence for base balance
gtcacttaag

SEQ ID NO: 332 - DNA, 10 bp, Artificial Sequence
  tag sequence for base balance
tggctcgctt

SEQ ID NO: 333 - DNA, 10 bp, Artificial Sequence
  tag sequence for base balance
cgcactatgg

SEQ ID NO: 334 - DNA, 10 bp, Artificial Sequence
  tag sequence for base balance
gaggtacatt

SEQ ID NO: 335 - DNA, 10 bp, Artificial Sequence
  tag sequence for base balance
tcttagtgac

SEQ ID NO: 336 - DNA, 10 bp, Artificial Sequence
  tag sequence for base balance
atacgcgcca

SEQ ID NO: 337 - DNA, 10 bp, Artificial Sequence
  tag sequence for base balance
tgaggcatat

SEQ ID NO: 338 - DNA, 10 bp, Artificial Sequence
  tag sequence for base balance
attctatggc

SEQ ID NO: 339 - DNA, 10 bp, Artificial Sequence
  tag sequence for base balance
ccgtagcatg

SEQ ID NO: 340 - DNA, 10 bp, Artificial Sequence
  tag sequence for base balance
gacactgcca

SEQ ID NO: 341 - DNA, 10 bp, Artificial Sequence
  tag sequence for base balance
acggcattaa

SEQ ID NO: 342 - DNA, 10 bp, Artificial Sequence
  tag sequence for base balance
gtaagcgagg

SEQ ID NO: 343 - DNA, 10 bp, Artificial Sequence
  tag sequence for base balance
cattatcgct

SEQ ID NO: 344 - DNA, 10 bp, Artificial Sequence
  tag sequence for base balance
tgcctgactc

SEQ ID NO: 345 - DNA, 10 bp, Artificial Sequence
  tag sequence for base balance
gactcatcca

SEQ ID NO: 346 - DNA, 10 bp, Artificial Sequence
  tag sequence for base balance
acgaacatac

SEQ ID NO: 347 - DNA, 10 bp, Artificial Sequence
  tag sequence for base balance
ttacgtcggt

SEQ ID NO: 348 - DNA, 10 bp, Artificial Sequence
  tag sequence for base balance
cgtgtggatg

SEQ ID NO: 349 - DNA, 10 bp, Artificial Sequence
  tag sequence for base balance
cctaacattc

SEQ ID NO: 350 - DNA, 10 bp, Artificial Sequence
  tag sequence for base balance
atcgcacgca

SEQ ID NO: 351 - DNA, 10 bp, Artificial Sequence
  tag sequence for base balance
gaacgttcgg

SEQ ID NO: 352 - DNA, 10 bp, Artificial Sequence
  tag sequence for base balance
tggttggaat

SEQ ID NO: 353 - DNA, 10 bp, Artificial Sequence
  tag sequence for base balance
aacaagtgag

SEQ ID NO: 354 - DNA, 10 bp, Artificial Sequence
  tag sequence for base balance
gttgttgctc

SEQ ID NO: 355 - DNA, 10 bp, Artificial Sequence
  tag sequence for base balance
tgacgcaact

SEQ ID NO: 356 - DNA, 10 bp, Artificial Sequence
  tag sequence for base balance
ccgtcactga

SEQ ID NO: 357 - DNA, 10 bp, Artificial Sequence
  tag sequence for base balance
tgttattccg

SEQ ID NO: 358 - DNA, 10 bp, Artificial Sequence
  tag sequence for base balance
ctactcaaga

SEQ ID NO: 359 - DNA, 10 bp, Artificial Sequence
  tag sequence for base balance
acgagacgtc

SEQ ID NO: 360 - DNA, 10 bp, Artificial Sequence
  tag sequence for base balance
gacgcggtat

SEQ ID NO: 361 - DNA, 10 bp, Artificial Sequence
  tag sequence for base balance
gatgacgtta

SEQ ID NO: 362 - DNA, 10 bp, Artificial Sequence
  tag sequence for base balance
agccgatacc

SEQ ID NO: 363 - DNA, 10 bp, Artificial Sequence
  tag sequence for base balance
ttatctcgag

SEQ ID NO: 364 - DNA, 10 bp, Artificial Sequence
  tag sequence for base balance
ccgatgacgt

SEQ ID NO: 365 - DNA, 10 bp, Artificial Sequence
  tag sequence for base balance
gtgagttcgc

SEQ ID NO: 366 - DNA, 10 bp, Artificial Sequence
  tag sequence for base balance
acacaacatg

SEQ ID NO: 367 - DNA, 10 bp, Artificial Sequence
  tag sequence for base balance
cgtgcgatca

SEQ ID NO: 368 - DNA, 10 bp, Artificial Sequence
  tag sequence for base balance
tacttcggat

SEQ ID NO: 369 - DNA, 10 bp, Artificial Sequence
  tag sequence for base balance
ctatcggtgt

SEQ ID NO: 370 - DNA, 10 bp, Artificial Sequence
  tag sequence for base balance
gacattcaag

SEQ ID NO: 371 - DNA, 10 bp, Artificial Sequence
  tag sequence for base balance
agtgacacca

SEQ ID NO: 372 - DNA, 10 bp, Artificial Sequence
  tag sequence for base balance
tcgcgatgtc

SEQ ID NO: 373 - DNA, 10 bp, Artificial Sequence
  tag sequence for base balance
cgagtcagtc

SEQ ID NO: 374 - DNA, 10 bp, Artificial Sequence
  tag sequence for base balance
ttgccagtga

SEQ ID NO: 375 - DNA, 10 bp, Artificial Sequence
  tag sequence for base balance
aacaagcact

SEQ ID NO: 376 - DNA, 10 bp, Artificial Sequence
  tag sequence for base balance
gcttgttcag

SEQ ID NO: 377 - DNA, 10 bp, Artificial Sequence
  tag sequence for base balance
tagtgatgtg

SEQ ID NO: 378 - DNA, 10 bp, Artificial Sequence
  tag sequence for base balance
cgtgtgacat

SEQ ID NO: 379 - DNA, 10 bp, Artificial Sequence
  tag sequence for base balance
atccaccacc

SEQ ID NO: 380 - DNA, 10 bp, Artificial Sequence
  tag sequence for base balance
gcaactgtga

SEQ ID NO: 381 - DNA, 10 bp, Artificial Sequence
  tag sequence for base balance
atccaccggt

SEQ ID NO: 382 - DNA, 10 bp, Artificial Sequence
  tag sequence for base balance
ccgtcattac

SEQ ID NO: 383 - DNA, 10 bp, Artificial Sequence
  tag sequence for base balance
tgagtggcta

SEQ ID NO: 384 - DNA, 10 bp, Artificial Sequence
  tag sequence for base balance
gatagtaacg

SEQ ID NO: 385 - DNA, 10 bp, Artificial Sequence
  tag sequence for base balance
cgtcgatgac

SEQ ID NO: 386 - DNA, 10 bp, Artificial Sequence
  tag sequence for base balance
atataaggcg

SEQ ID NO: 387 - DNA, 10 bp, Artificial Sequence
  tag sequence for base balance
gatcgtgctc

SEQ ID NO: 388 - DNA, 10 bp, Artificial Sequence
  tag sequence for base balance
cagtcttcgg

SEQ ID NO: 389 - DNA, 10 bp, Artificial Sequence
  tag sequence for base balance
agaacgatct

SEQ ID NO: 390 - DNA, 10 bp, Artificial Sequence
  tag sequence for base balance
ttggtgcatt

SEQ ID NO: 391 - DNA, 10 bp, Artificial Sequence
  tag sequence for base balance
gccgtcataa

SEQ ID NO: 392 - DNA, 10 bp, Artificial Sequence
  tag sequence for base balance
tccaaccaga

SEQ ID NO: 393 - DNA, 10 bp, Artificial Sequence
  tag sequence for base balance
gatagcaaga

SEQ ID NO: 394 - DNA, 10 bp, Artificial Sequence
  tag sequence for base balance
accgtgcttc

SEQ ID NO: 395 - DNA, 10 bp, Artificial Sequence
  tag sequence for base balance
gcagatgtaa

SEQ ID NO: 396 - DNA, 10 bp, Artificial Sequence
  tag sequence for base balance
tgttggagcg

SEQ ID NO: 397 - DNA, 10 bp, Artificial Sequence
  tag sequence for base balance
ttgtatccac

SEQ ID NO: 398 - DNA, 10 bp, Artificial Sequence
  tag sequence for base balance
cgcacagatg

SEQ ID NO: 399 - DNA, 10 bp, Artificial Sequence
  tag sequence for base balance
caactctcgt

SEQ ID NO: 400 - DNA, 10 bp, Artificial Sequence
  tag sequence for base balance
atgccatgct

SEQ ID NO: 401 - DNA, 10 bp, Artificial Sequence
  tag sequence for base balance
aggcagctta

SEQ ID NO: 402 - DNA, 10 bp, Artificial Sequence
  tag sequence for base balance
tagcctagcg

SEQ ID NO: 403 - DNA, 10 bp, Artificial Sequence
  tag sequence for base balance
atcacgtgcg

SEQ ID NO: 404 - DNA, 10 bp, Artificial Sequence
  tag sequence for base balance
cgttatgcgc

SEQ ID NO: 405 - DNA, 10 bp, Artificial Sequence
  tag sequence for base balance
caaggatcga

SEQ ID NO: 406 - DNA, 10 bp, Artificial Sequence
  tag sequence for base balance
gttgtcgtat

SEQ ID NO: 407 - DNA, 10 bp, Artificial Sequence
  tag sequence for base balance
gcaatcaatc

SEQ ID NO: 408 - DNA, 10 bp, Artificial Sequence
  tag sequence for base balance
tcctgacaat

SEQ ID NO: 409 - DNA, 10 bp, Artificial Sequence
  tag sequence for base balance
aggtgcctta

SEQ ID NO: 410 - DNA, 10 bp, Artificial Sequence
  tag sequence for base balance
aagaaccaag

SEQ ID NO: 411 - DNA, 10 bp, Artificial Sequence
  tag sequence for base balance
catacatgac

SEQ ID NO: 412 - DNA, 10 bp, Artificial Sequence
  tag sequence for base balance
tgcctggtga

SEQ ID NO: 413 - DNA, 10 bp, Artificial Sequence
  tag sequence for base balance
tctcggagtt

SEQ ID NO: 414 - DNA, 10 bp, Artificial Sequence
  tag sequence for base balance
gtcgtagact

SEQ ID NO: 415 - DNA, 10 bp, Artificial Sequence
  tag sequence for base balance
gcatataccg

SEQ ID NO: 416 - DNA, 10 bp, Artificial Sequence
  tag sequence for base balance
ctagcttcgc

SEQ ID NO: 417 - DNA, 10 bp, Artificial Sequence
  tag sequence for base balance
gacgtatcaa

SEQ ID NO: 418 - DNA, 10 bp, Artificial Sequence
  tag sequence for base balance
cctgctagga

SEQ ID NO: 419 - DNA, 10 bp, Artificial Sequence
  tag sequence for base balance
caacttggcg

SEQ ID NO: 420 - DNA, 10 bp, Artificial Sequence
  tag sequence for base balance
atgcgacctc

SEQ ID NO: 421 - DNA, 10 bp, Artificial Sequence
  tag sequence for base balance
gttaaggacg

SEQ ID NO: 422 - DNA, 10 bp, Artificial Sequence
  tag sequence for base balance
tccaagttgt

SEQ ID NO: 423 - DNA, 10 bp, Artificial Sequence
  tag sequence for base balance
aggtccattc

SEQ ID NO: 424 - DNA, 10 bp, Artificial Sequence
  tag sequence for base balance
tgatgccaat

SEQ ID NO: 425 - DNA, 10 bp, Artificial Sequence
  tag sequence for base balance
cctaagagtt

SEQ ID NO: 426 - DNA, 10 bp, Artificial Sequence
  tag sequence for base balance
gatactagct

SEQ ID NO: 427 - DNA, 10 bp, Artificial Sequence
  tag sequence for base balance
aaggcatctc

SEQ ID NO: 428 - DNA, 10 bp, Artificial Sequence
  tag sequence for base balance
tggcatctgg

SEQ ID NO: 429 - DNA, 10 bp, Artificial Sequence
  tag sequence for base balance
acactggagc

SEQ ID NO: 430 - DNA, 10 bp, Artificial Sequence
  tag sequence for base balance
gtatgccaca

SEQ ID NO: 431 - DNA, 10 bp, Artificial Sequence
  tag sequence for base balance
ctcttagtag

SEQ ID NO: 432 - DNA, 10 bp, Artificial Sequence
  tag sequence for base balance
tgcggctcaa

SEQ ID NO: 433 - DNA, 10 bp, Artificial Sequence
  tag sequence for base balance
taaggctaga

SEQ ID NO: 434 - DNA, 10 bp, Artificial Sequence
  tag sequence for base balance
gaggagataa

SEQ ID NO: 435 - DNA, 10 bp, Artificial Sequence
  tag sequence for base balance
cgttagcact

SEQ ID NO: 436 - DNA, 10 bp, Artificial Sequence
  tag sequence for base balance
aggctaggat

SEQ ID NO: 437 - DNA, 10 bp, Artificial Sequence
  tag sequence for base balance
atctcactgg

SEQ ID NO: 438 - DNA, 10 bp, Artificial Sequence
  tag sequence for base balance
tccagttctc

SEQ ID NO: 439 - DNA, 10 bp, Artificial Sequence
  tag sequence for base balance
gtaaccgctc

SEQ ID NO: 440 - DNA, 10 bp, Artificial Sequence
  tag sequence for base balance
cctcttagcg

SEQ ID NO: 441 - DNA, 10 bp, Artificial Sequence
  tag sequence for base balance
actaaggctg

SEQ ID NO: 442 - DNA, 10 bp, Artificial Sequence
  tag sequence for base balance
gaggagtatg

SEQ ID NO: 443 - DNA, 10 bp, Artificial Sequence
  tag sequence for base balance
tagtcaacgc

SEQ ID NO: 444 - DNA, 10 bp, Artificial Sequence
  tag sequence for base balance
ccatcaagga

SEQ ID NO: 445 - DNA, 10 bp, Artificial Sequence
  tag sequence for base balance
agcctctgct

SEQ ID NO: 446 - DNA, 10 bp, Artificial Sequence
  tag sequence for base balance
ttcgttcaca

SEQ ID NO: 447 - DNA, 10 bp, Artificial Sequence
  tag sequence for base balance
gtaagtgtac

SEQ ID NO: 448 - DNA, 10 bp, Artificial Sequence
  tag sequence for base balance
cgtcgcctat

SEQ ID NO: 449 - DNA, 10 bp, Artificial Sequence
  tag sequence for base balance
tgtgaaggag

SEQ ID NO: 450 - DNA, 10 bp, Artificial Sequence
  tag sequence for base balance
agaccggttc

SEQ ID NO: 451 - DNA, 10 bp, Artificial Sequence
  tag sequence for base balance
ctcgcctaac

SEQ ID NO: 452 - DNA, 10 bp, Artificial Sequence
  tag sequence for base balance
ccgctgtcta

SEQ ID NO: 453 - DNA, 10 bp, Artificial Sequence
  tag sequence for base balance
gacataacct

SEQ ID NO: 454 - DNA, 10 bp, Artificial Sequence
  tag sequence for base balance
gagaatagca

SEQ ID NO: 455 - DNA, 10 bp, Artificial Sequence
  tag sequence for base balance
ttatgtcagg

SEQ ID NO: 456 - DNA, 10 bp, Artificial Sequence
  tag sequence for base balance
acttgcctgt

SEQ ID NO: 457 - DNA, 10 bp, Artificial Sequence
  tag sequence for base balance
tgctggatct

SEQ ID NO: 458 - DNA, 10 bp, Artificial Sequence
  tag sequence for base balance
agtagtgttg

SEQ ID NO: 459 - DNA, 10 bp, Artificial Sequence
  tag sequence for base balance
ctccaatcct

SEQ ID NO: 460 - DNA, 10 bp, Artificial Sequence
  tag sequence for base balance
cctatgtgta

SEQ ID NO: 461 - DNA, 10 bp, Artificial Sequence
  tag sequence for base balance
taatatccgc

SEQ ID NO: 462 - DNA, 10 bp, Artificial Sequence
  tag sequence for base balance
gtgccacaac

SEQ ID NO: 463 - DNA, 10 bp, Artificial Sequence
  tag sequence for base balance
gcagtcagga

SEQ ID NO: 464 - DNA, 10 bp, Artificial Sequence
  tag sequence for base balance
aaggccgaag

SEQ ID NO: 465 - DNA, 10 bp, Artificial Sequence
  tag sequence for base balance
cgtgttagag

SEQ ID NO: 466 - DNA, 10 bp, Artificial Sequence
  tag sequence for base balance
acaggacgat

SEQ ID NO: 467 - DNA, 10 bp, Artificial Sequence
  tag sequence for base balance
cggataacgg

SEQ ID NO: 468 - DNA, 10 bp, Artificial Sequence
  tag sequence for base balance
gttagtgcct

SEQ ID NO: 469 - DNA, 10 bp, Artificial Sequence
  tag sequence for base balance
aagcacgaca

SEQ ID NO: 470 - DNA, 10 bp, Artificial Sequence
  tag sequence for base balance
gtctccttgc

SEQ ID NO: 471 - DNA, 10 bp, Artificial Sequence
  tag sequence for base balance
tcaccgtata

SEQ ID NO: 472 - DNA, 10 bp, Artificial Sequence
  tag sequence for base balance
tactagcttc

SEQ ID NO: 473 - DNA, 10 bp, Artificial Sequence
  tag sequence for base balance
accattcacg

SEQ ID NO: 474 - DNA, 10 bp, Artificial Sequence
  tag sequence for base balance
agtagctagt

SEQ ID NO: 475 - DNA, 10 bp, Artificial Sequence
  tag sequence for base balance
ttccgaaggt

SEQ ID NO: 476 - DNA, 10 bp, Artificial Sequence
  tag sequence for base balance
gtaccaacca

SEQ ID NO: 477 - DNA, 10 bp, Artificial Sequence
  tag sequence for base balance
tatgtgtgtc

SEQ ID NO: 478 - DNA, 10 bp, Artificial Sequence
  tag sequence for base balance
cgatcggtac

SEQ ID NO: 479 - DNA, 10 bp, Artificial Sequence
  tag sequence for base balance
gaggatctag

SEQ ID NO: 480 - DNA, 10 bp, Artificial Sequence
  tag sequence for base balance
ccgtacgcta

SEQ ID NO: 481 - DNA, 10 bp, Artificial Sequence
  tag sequence for base balance
gtcaactcgg

SEQ ID NO: 482 - DNA, 10 bp, Artificial Sequence
  tag sequence for base balance
tggaagcaca

SEQ ID NO: 483 - DNA, 10 bp, Artificial Sequence
  tag sequence for base balance
tcgttgtagc

SEQ ID NO: 484 - DNA, 10 bp, Artificial Sequence
  tag sequence for base balance
agccgaagtt

SEQ ID NO: 485 - DNA, 10 bp, Artificial Sequence
  tag sequence for base balance
gatgcaacct

SEQ ID NO: 486 - DNA, 10 bp, Artificial Sequence
  tag sequence for base balance
acttccgttc

SEQ ID NO: 487 - DNA, 10 bp, Artificial Sequence
  tag sequence for base balance
caagttggaa

SEQ ID NO: 488 - DNA, 10 bp, Artificial Sequence
  tag sequence for base balance
ctacgtctag

SEQ ID NO: 489 - DNA, 10 bp, Artificial Sequence
  tag sequence for base balance
tgtcgttaag

SEQ ID NO: 490 - DNA, 10 bp, Artificial Sequence
  tag sequence for base balance
gtctcaatga

SEQ ID NO: 491 - DNA, 10 bp, Artificial Sequence
  tag sequence for base balance
gatcaagcca

SEQ ID NO: 492 - DNA, 10 bp, Artificial Sequence
  tag sequence for base balance
aactccgatc

SEQ ID NO: 493 - DNA, 10 bp, Artificial Sequence
  tag sequence for base balance
ctaagtcttc

SEQ ID NO: 494 - DNA, 10 bp, Artificial Sequence
  tag sequence for base balance
tgagagcgct

SEQ ID NO: 495 - DNA, 10 bp, Artificial Sequence
  tag sequence for base balance
acgatctcgg

SEQ ID NO: 496 - DNA, 10 bp, Artificial Sequence
  tag sequence for base balance
ccggtgagat

SEQ ID NO: 497 - DNA, 10 bp, Artificial Sequence
  tag sequence for base balance
catctcatga

SEQ ID NO: 498 - DNA, 10 bp, Artificial Sequence
  tag sequence for base balance
ctgtgactcg

SEQ ID NO: 499 - DNA, 10 bp, Artificial Sequence
  tag sequence for base balance
agaccttgga

SEQ ID NO: 500 - DNA, 10 bp, Artificial Sequence
  tag sequence for base balance
actgtcgacg

SEQ ID NO: 501 - DNA, 10 bp, Artificial Sequence
  tag sequence for base balance
gtgaagacac

SEQ ID NO: 502 - DNA, 10 bp, Artificial Sequence
  tag sequence for base balance
tactgtgcat

SEQ ID NO: 503 - DNA, 10 bp, Artificial Sequence
  tag sequence for base balance
gcagagcgtt

SEQ ID NO: 504 - DNA, 10 bp, Artificial Sequence
  tag sequence for base balance
tgcacatatc

SEQ ID NO: 505 - DNA, 10 bp, Artificial Sequence
  tag sequence for base balance
cctgtgtaac

SEQ ID NO: 506 - DNA, 10 bp, Artificial Sequence
  tag sequence for base balance
aacgatgcca

SEQ ID NO: 507 - DNA, 10 bp, Artificial Sequence
  tag sequence for base balance
tcgagcatag

SEQ ID NO: 508 - DNA, 10 bp, Artificial Sequence
  tag sequence for base balance
gaattcctgt

SEQ ID NO: 509 - DNA, 10 bp, Artificial Sequence
  tag sequence for base balance
agaacgtccg

SEQ ID NO: 510 - DNA, 10 bp, Artificial Sequence
  tag sequence for base balance
cgccaaggta

SEQ ID NO: 511 - DNA, 10 bp, Artificial Sequence
  tag sequence for base balance
ttgtgaaggc

SEQ ID NO: 512 - DNA, 10 bp, Artificial Sequence
  tag sequence for base balance
gttcctcatt

SEQ ID NO: 513 - DNA, 10 bp, Artificial Sequence
  tag sequence for base balance
cgtcactatt

SEQ ID NO: 514 - DNA, 10 bp, Artificial Sequence
  tag sequence for base balance
gcgatgcaga

SEQ ID NO: 515 - DNA, 10 bp, Artificial Sequence
  tag sequence for base balance
cgtgtcctag

SEQ ID NO: 516 - DNA, 10 bp, Artificial Sequence
  tag sequence for base balance
gaagaatgga

SEQ ID NO: 517 - DNA, 10 bp, Artificial Sequence
  tag sequence for base balance
atgtgtggct

SEQ ID NO: 518 - DNA, 10 bp, Artificial Sequence
  tag sequence for base balance
tcctgtacac

SEQ ID NO: 519 - DNA, 10 bp, Artificial Sequence
  tag sequence for base balance
ttaccgattc

SEQ ID NO: 520 - DNA, 10 bp, Artificial Sequence
  tag sequence for base balance
aacacagccg

SEQ ID NO: 521 - DNA, 10 bp, Artificial Sequence
  tag sequence for base balance
tcataccaag

SEQ ID NO: 522 - DNA, 10 bp, Artificial Sequence
  tag sequence for base balance
gaagcttact

SEQ ID NO: 523 - DNA, 10 bp, Artificial Sequence
  tag sequence for base balance
gtcagtaggt

SEQ ID NO: 524 - DNA, 10 bp, Artificial Sequence
  tag sequence for base balance
ctgtgcgtag

SEQ ID NO: 525 - DNA, 10 bp, Artificial Sequence
  tag sequence for base balance
agtgaatgga

SEQ ID NO: 526 - DNA, 10 bp, Artificial Sequence
  tag sequence for base balance
tccacggttc

SEQ ID NO: 527 - DNA, 10 bp, Artificial Sequence
  tag sequence for base balance
aatctgcctc

SEQ ID NO: 528 - DNA, 10 bp, Artificial Sequence
  tag sequence for base balance
cggctaacca

SEQ ID NO: 529 - DNA, 10 bp, Artificial Sequence
  tag sequence for base balance
gacatacagt

SEQ ID NO: 530 - DNA, 10 bp, Artificial Sequence
  tag sequence for base balance
agaggcctca

SEQ ID NO: 531 - DNA, 10 bp, Artificial Sequence
  tag sequence for base balance
cctgatattg

SEQ ID NO: 532 - DNA, 10 bp, Artificial Sequence
  tag sequence for base balance
gtgctgaact

SEQ ID NO: 533 - DNA, 10 bp, Artificial Sequence
  tag sequence for base balance
cgatggtcag

SEQ ID NO: 534 - DNA, 10 bp, Artificial Sequence
  tag sequence for base balance
aatacagcgc

SEQ ID NO: 535 - DNA, 10 bp, Artificial Sequence
  tag sequence for base balance
tcgtcctgac

SEQ ID NO: 536 - DNA, 10 bp, Artificial Sequence
  tag sequence for base balance
ttccatggta

SEQ ID NO: 537 - DNA, 10 bp, Artificial Sequence
  tag sequence for base balance
cgttcgactg

SEQ ID NO: 538 - DNA, 10 bp, Artificial Sequence
  tag sequence for base balance
cgctacactc

SEQ ID NO: 539 - DNA, 10 bp, Artificial Sequence
  tag sequence for base balance
atagatcggt

SEQ ID NO: 540 - DNA, 10 bp, Artificial Sequence
  tag sequence for base balance
accagattca

SEQ ID NO: 541 - DNA, 10 bp, Artificial Sequence
  tag sequence for base balance
gatacggagg

SEQ ID NO: 542 - DNA, 10 bp, Artificial Sequence
  tag sequence for base balance
ttaggaggaa

SEQ ID NO: 543 - DNA, 10 bp, Artificial Sequence
  tag sequence for base balance
gcgcttctac

SEQ ID NO: 544 - DNA, 10 bp, Artificial Sequence
  tag sequence for base balance
tagctctact

SEQ ID NO: 545 - DNA, 10 bp, Artificial Sequence
  tag sequence for base balance
ccacctgctt

SEQ ID NO: 546 - DNA, 10 bp, Artificial Sequence
  tag sequence for base balance
cgtaggtctg

SEQ ID NO: 547 - DNA, 10 bp, Artificial Sequence
  tag sequence for base balance
tcttagtgac

SEQ ID NO: 548 - DNA, 10 bp, Artificial Sequence
  tag sequence for base balance
atcctagaac

SEQ ID NO: 549 - DNA, 10 bp, Artificial Sequence
  tag sequence for base balance
aggtgtaggt

SEQ ID NO: 550 - DNA, 10 bp, Artificial Sequence
  tag sequence for base balance
gtaaccatca

SEQ ID NO: 551 - DNA, 10 bp, Artificial Sequence
  tag sequence for base balance
taggtccaga

SEQ ID NO: 552 - DNA, 10 bp, Artificial Sequence
  tag sequence for base balance
gacgaactcg

SEQ ID NO: 553 - DNA, 10 bp, Artificial Sequence
  tag sequence for base balance
ccaacagatt

SEQ ID NO: 554 - DNA, 10 bp, Artificial Sequence
  tag sequence for base balance
cctctatctc

SEQ ID NO: 555 - DNA, 10 bp, Artificial Sequence
  tag sequence for base balance
gagtgtctca

SEQ ID NO: 556 - DNA, 10 bp, Artificial Sequence
  tag sequence for base balance
ttggcttaag

SEQ ID NO: 557 - DNA, 10 bp, Artificial Sequence
  tag sequence for base balance
agtcgcatgg

SEQ ID NO: 558 - DNA, 10 bp, Artificial Sequence
  tag sequence for base balance
atcttgcggc

SEQ ID NO: 559 - DNA, 10 bp, Artificial Sequence
  tag sequence for base balance
tgcaaggcct

SEQ ID NO: 560 - DNA, 10 bp, Artificial Sequence
  tag sequence for base balance
gaagacagaa

SEQ ID NO: 561 - DNA, 10 bp, Artificial Sequence
  tag sequence for base balance
acagactcat

SEQ ID NO: 562 - DNA, 10 bp, Artificial Sequence
  tag sequence for base balance
gagttgaggc

SEQ ID NO: 563 - DNA, 10 bp, Artificial Sequence
  tag sequence for base balance
gttctagacg

SEQ ID NO: 564 - DNA, 10 bp, Artificial Sequence
  tag sequence for base balance
agttcaggcg

SEQ ID NO: 565 - DNA, 10 bp, Artificial Sequence
  tag sequence for base balance
cagacgatga

SEQ ID NO: 566 - DNA, 10 bp, Artificial Sequence
  tag sequence for base balance
tccaacctat

SEQ ID NO: 567 - DNA, 10 bp, Artificial Sequence
  tag sequence for base balance
ttacgttctc

SEQ ID NO: 568 - DNA, 10 bp, Artificial Sequence
  tag sequence for base balance
cgcggtcata

SEQ ID NO: 569 - DNA, 10 bp, Artificial Sequence
  tag sequence for base balance
aatccttccg

SEQ ID NO: 570 - DNA, 10 bp, Artificial Sequence
  tag sequence for base balance
gaggattgaa

SEQ ID NO: 571 - DNA, 10 bp, Artificial Sequence
  tag sequence for base balance
atcggagtgc

SEQ ID NO: 572 - DNA, 10 bp, Artificial Sequence
  tag sequence for base balance
gcgttaagtg

SEQ ID NO: 573 - DNA, 10 bp, Artificial Sequence
  tag sequence for base balance
cctcggcaat

SEQ ID NO: 574 - DNA, 10 bp, Artificial Sequence
  tag sequence for base balance
ttaacggctt

SEQ ID NO: 575 - DNA, 10 bp, Artificial Sequence
  tag sequence for base balance
tgattccaca

SEQ ID NO: 576 - DNA, 10 bp, Artificial Sequence
  tag sequence for base balance
cgcaacatgc

SEQ ID NO: 577 - DNA, 10 bp, Artificial Sequence
  tag sequence for base balance
tcaatgaggt

SEQ ID NO: 578 - DNA, 10 bp, Artificial Sequence
  tag sequence for base balance
gtccaagctg

SEQ ID NO: 579 - DNA, 10 bp, Artificial Sequence
  tag sequence for base balance
gcgcactaag

SEQ ID NO: 580 - DNA, 10 bp, Artificial Sequence
  tag sequence for base balance
cggtgagtga

SEQ ID NO: 581 - DNA, 10 bp, Artificial Sequence
  tag sequence for base balance
tgaatccgac

SEQ ID NO: 582 - DNA, 10 bp, Artificial Sequence
  tag sequence for base balance
aatgctcact

SEQ ID NO: 583 - DNA, 10 bp, Artificial Sequence
  tag sequence for base balance
atcggttcca

SEQ ID NO: 584 - DNA, 10 bp, Artificial Sequence
  tag sequence for base balance
cattcgattc

SEQ ID NO: 585 - DNA, 10 bp, Artificial Sequence
  tag sequence for base balance
ttatgcctac

SEQ ID NO: 586 - DNA, 10 bp, Artificial Sequence
  tag sequence for base balance
atcctccgac

SEQ ID NO: 587 - DNA, 10 bp, Artificial Sequence
  tag sequence for base balance
cgcagatata

SEQ ID NO: 588 - DNA, 10 bp, Artificial Sequence
  tag sequence for base balance
acaacatgtg

SEQ ID NO: 589 - DNA, 10 bp, Artificial Sequence
  tag sequence for base balance
tcggatgagg

SEQ ID NO: 590 - DNA, 10 bp, Artificial Sequence
  tag sequence for base balance
cgtgcgatca

SEQ ID NO: 591 - DNA, 10 bp, Artificial Sequence
  tag sequence for base balance
gatcttacgt

SEQ ID NO: 592 - DNA, 10 bp, Artificial Sequence
  tag sequence for base balance
gagtaggcct

SEQ ID NO: 593 - DNA, 10 bp, Artificial Sequence
  tag sequence for base balance
aagagagaag

SEQ ID NO: 594 - DNA, 10 bp, Artificial Sequence
  tag sequence for base balance
gttctcacgg

SEQ ID NO: 595 - DNA, 10 bp, Artificial Sequence
  tag sequence for base balance
aagcggtgaa

SEQ ID NO: 596 - DNA, 10 bp, Artificial Sequence
  tag sequence for base balance
cgctcagtta

SEQ ID NO: 597 - DNA, 10 bp, Artificial Sequence
  tag sequence for base balance
ctagacactc

SEQ ID NO: 598 - DNA, 10 bp, Artificial Sequence
  tag sequence for base balance
gcttattgct

SEQ ID NO: 599 - DNA, 10 bp, Artificial Sequence
  tag sequence for base balance
tgcgttctgc

SEQ ID NO: 600 - DNA, 10 bp, Artificial Sequence
  tag sequence for base balance
tcaacgcact

SEQ ID NO: 601 - DNA, 10 bp, Artificial Sequence
  tag sequence for base balance
cttgcttaca

SEQ ID NO: 602 - DNA, 10 bp, Artificial Sequence
  tag sequence for base balance
ctcctcgcaa

SEQ ID NO: 603 - DNA, 10 bp, Artificial Sequence
  tag sequence for base balance
tctcagcctc

SEQ ID NO: 604 - DNA, 10 bp, Artificial Sequence
  tag sequence for base balance
gacggagtac

SEQ ID NO: 605 - DNA, 10 bp, Artificial Sequence
  tag sequence for base balance
acgtctatct

SEQ ID NO: 606 - DNA, 10 bp, Artificial Sequence
  tag sequence for base balance
gaatagagtg

SEQ ID NO: 607 - DNA, 10 bp, Artificial Sequence
  tag sequence for base balance
aggatacggt

SEQ ID NO: 608 - DNA, 10 bp, Artificial Sequence
  tag sequence for base balance
tgaagctagg

SEQ ID NO: 609 - DNA, 10 bp, Artificial Sequence
  tag sequence for base balance
aacctcgcac

SEQ ID NO: 610 - DNA, 10 bp, Artificial Sequence
  tag sequence for base balance
gatccggact

SEQ ID NO: 611 - DNA, 10 bp, Artificial Sequence
  tag sequence for base balance
gtgtctacag

SEQ ID NO: 612 - DNA, 10 bp, Artificial Sequence
  tag sequence for base balance
acggagaatc

SEQ ID NO: 613 - DNA, 10 bp, Artificial Sequence
  tag sequence for base balance
tgttacctca

SEQ ID NO: 614 - DNA, 10 bp, Artificial Sequence
  tag sequence for base balance
tcaatacgtg

SEQ ID NO: 615 - DNA, 10 bp, Artificial Sequence
  tag sequence for base balance
ctaagttggt

SEQ ID NO: 616 - DNA, 10 bp, Artificial Sequence
  tag sequence for base balance
cgcggattga

SEQ ID NO: 617 - DNA, 10 bp, Artificial Sequence
  tag sequence for base balance
acctcacata

SEQ ID NO: 618 - DNA, 10 bp, Artificial Sequence
  tag sequence for base balance
cggactgtct

SEQ ID NO: 619 - DNA, 10 bp, Artificial Sequence
  tag sequence for base balance
cgtggcagaa

SEQ ID NO: 620 - DNA, 10 bp, Artificial Sequence
  tag sequence for base balance
attcgaatgc

SEQ ID NO: 621 - DNA, 10 bp, Artificial Sequence
  tag sequence for base balance
taggttcaag

SEQ ID NO: 622 - DNA, 10 bp, Artificial Sequence
  tag sequence for base balance
gtccactctc

SEQ ID NO: 623 - DNA, 10 bp, Artificial Sequence
  tag sequence for base balance
gaatagtgct

SEQ ID NO: 624 - DNA, 10 bp, Artificial Sequence
  tag sequence for base balance
tcaatggcgg

SEQ ID NO: 625 - DNA, 10 bp, Artificial Sequence
  tag sequence for base balance
aagcatcctg

SEQ ID NO: 626 - DNA, 10 bp, Artificial Sequence
  tag sequence for base balance
gtggtgttca

SEQ ID NO: 627 - DNA, 10 bp, Artificial Sequence
  tag sequence for base balance
gtacaacgtt

SEQ ID NO: 628 - DNA, 10 bp, Artificial Sequence
  tag sequence for base balance
cctgcagcat

SEQ ID NO: 629 - DNA, 10 bp, Artificial Sequence
  tag sequence for base balance
tctatcgtac

SEQ ID NO: 630 - DNA, 10 bp, Artificial Sequence
  tag sequence for base balance
cactcgtagc

SEQ ID NO: 631 - DNA, 10 bp, Artificial Sequence
  tag sequence for base balance
tgcagcagca

SEQ ID NO: 632 - DNA, 10 bp, Artificial Sequence
  tag sequence for base balance
agatgtaagg

SEQ ID NO: 633 - DNA, 10 bp, Artificial Sequence
  tag sequence for base balance
gtgtaaccgc

SEQ ID NO: 634 - DNA, 10 bp, Artificial Sequence
  tag sequence for base balance
catcggaaga

SEQ ID NO: 635 - DNA, 10 bp, Artificial Sequence
  tag sequence for base balance
gaggaattac

SEQ ID NO: 636 - DNA, 10 bp, Artificial Sequence
  tag sequence for base balance
tgttcgctct

SEQ ID NO: 637 - DNA, 10 bp, Artificial Sequence
  tag sequence for base balance
accgtctata

SEQ ID NO: 638 - DNA, 10 bp, Artificial Sequence
  tag sequence for base balance
tgcctcagag

SEQ ID NO: 639 - DNA, 10 bp, Artificial Sequence
  tag sequence for base balance
acaagtgccg

SEQ ID NO: 640 - DNA, 10 bp, Artificial Sequence
  tag sequence for base balance
ctaactggtt

SEQ ID NO: 641 - DNA, 10 bp, Artificial Sequence
  tag sequence for base balance
cagaacgtgg

SEQ ID NO: 642 - DNA, 10 bp, Artificial Sequence
  tag sequence for base balance
gttcttctgt

SEQ ID NO: 643 - DNA, 10 bp, Artificial Sequence
  tag sequence for base balance
cggtgaagtc

SEQ ID NO: 644 - DNA, 10 bp, Artificial Sequence
  tag sequence for base balance
gtaagatgag

SEQ ID NO: 645 - DNA, 10 bp, Artificial Sequence
  tag sequence for base balance
tgccatcaca

SEQ ID NO: 646 - DNA, 10 bp, Artificial Sequence
  tag sequence for base balance
tcctcggata

SEQ ID NO: 647 - DNA, 10 bp, Artificial Sequence
  tag sequence for base balance
acagtgtcct

SEQ ID NO: 648 - DNA, 10 bp, Artificial Sequence
  tag sequence for base balance
aatgccacac

SEQ ID NO: 649 - DNA, 10 bp, Artificial Sequence
  tag sequence for base balance
gaccactcga

SEQ ID NO: 650 - DNA, 10 bp, Artificial Sequence
  tag sequence for base balance
atggacaaca

SEQ ID NO: 651 - DNA, 10 bp, Artificial Sequence
  tag sequence for base balance
aacctacggt

SEQ ID NO: 652 - DNA, 10 bp, Artificial Sequence
  tag sequence for base balance
tctaggattg

SEQ ID NO: 653 - DNA, 10 bp, Artificial Sequence
  tag sequence for base balance
cggattctcg

SEQ ID NO: 654 - DNA, 10 bp, Artificial Sequence
  tag sequence for base balance
cgatcttctc

SEQ ID NO: 655 - DNA, 10 bp, Artificial Sequence
  tag sequence for base balance
tcatcggaat

SEQ ID NO: 656 - DNA, 10 bp, Artificial Sequence
  tag sequence for base balance
gttggaggac

SEQ ID NO: 657 - DNA, 10 bp, Artificial Sequence
  tag sequence for base balance
acacatgcta

SEQ ID NO: 658 - DNA, 10 bp, Artificial Sequence
  tag sequence for base balance
ccttaggacg

SEQ ID NO: 659 - DNA, 10 bp, Artificial Sequence
  tag sequence for base balance
ctaaccaatc

SEQ ID NO: 660 - DNA, 10 bp, Artificial Sequence
  tag sequence for base balance
aacgcacgag

SEQ ID NO: 661 - DNA, 10 bp, Artificial Sequence
  tag sequence for base balance
gtcggttcac

SEQ ID NO: 662 - DNA, 10 bp, Artificial Sequence
  tag sequence for base balance
tggctcatgt

SEQ ID NO: 663 - DNA, 10 bp, Artificial Sequence
  tag sequence for base balance
tggtggttgt

SEQ ID NO: 664 - DNA, 10 bp, Artificial Sequence
  tag sequence for base balance
gatatacgca

SEQ ID NO: 665 - DNA, 10 bp, Artificial Sequence
  tag sequence for base balance
cacgaacact

SEQ ID NO: 666 - DNA, 10 bp, Artificial Sequence
  tag sequence for base balance
tgcgtgagca

SEQ ID NO: 667 - DNA, 10 bp, Artificial Sequence
  tag sequence for base balance
gtactgacga

SEQ ID NO: 668 - DNA, 10 bp, Artificial Sequence
  tag sequence for base balance
ccgtgagttc

SEQ ID NO: 669 - DNA, 10 bp, Artificial Sequence
  tag sequence for base balance
acttacttgg

SEQ ID NO: 670 - DNA, 10 bp, Artificial Sequence
  tag sequence for base balance
agaagctcat

SEQ ID NO: 671 - DNA, 10 bp, Artificial Sequence
  tag sequence for base balance
gttactggac

SEQ ID NO: 672 - DNA, 10 bp, Artificial Sequence
  tag sequence for base balance
tagcctcatg

SEQ ID NO: 673 - DNA, 10 bp, Artificial Sequence
  tag sequence for base balance
tgacaatgac

SEQ ID NO: 674 - DNA, 10 bp, Artificial Sequence
  tag sequence for base balance
aatcacctct

SEQ ID NO: 675 - DNA, 10 bp, Artificial Sequence
  tag sequence for base balance
aacagacctg

SEQ ID NO: 676 - DNA, 10 bp, Artificial Sequence
  tag sequence for base balance
gctagtgtgt

SEQ ID NO: 677 - DNA, 10 bp, Artificial Sequence
  tag sequence for base balance
gcggttgata

SEQ ID NO: 678 - DNA, 10 bp, Artificial Sequence
  tag sequence for base balance
ctagcgacac

SEQ ID NO: 679 - DNA, 10 bp, Artificial Sequence
  tag sequence for base balance
cggttgaacg

SEQ ID NO: 680 - DNA, 10 bp, Artificial Sequence
  tag sequence for base balance
ttctcctgga

SEQ ID NO: 681 - DNA, 10 bp, Artificial Sequence
  tag sequence for base balance
agttagctgg

SEQ ID NO: 682 - DNA, 10 bp, Artificial Sequence
  tag sequence for base balance
agtcctgtaa

SEQ ID NO: 683 - DNA, 10 bp, Artificial Sequence
  tag sequence for base balance
taaggccggt

SEQ ID NO: 684 - DNA, 10 bp, Artificial Sequence
  tag sequence for base balance
gccttatcct

SEQ ID NO: 685 - DNA, 10 bp, Artificial Sequence
  tag sequence for base balance
cagcattcaa

SEQ ID NO: 686 - DNA, 10 bp, Artificial Sequence
  tag sequence for base balance
tcgagcaatc

SEQ ID NO: 687 - DNA, 10 bp, Artificial Sequence
  tag sequence for base balance
gtaacgaacg

SEQ ID NO: 688 - DNA, 10 bp, Artificial Sequence
  tag sequence for base balance
ctcgtaggtc

SEQ ID NO: 689 - DNA, 10 bp, Artificial Sequence
  tag sequence for base balance
ctggattact

SEQ ID NO: 690 - DNA, 10 bp, Artificial Sequence
  tag sequence for base balance
gtatgttcct

SEQ ID NO: 691 - DNA, 10 bp, Artificial Sequence
  tag sequence for base balance
tgttcgcgac

SEQ ID NO: 692 - DNA, 10 bp, Artificial Sequence
  tag sequence for base balance
cacggaattg

SEQ ID NO: 693 - DNA, 10 bp, Artificial Sequence
  tag sequence for base balance
agcacaatgc

SEQ ID NO: 694 - DNA, 10 bp, Artificial Sequence
  tag sequence for base balance
acacaccgga

SEQ ID NO: 695 - DNA, 10 bp, Artificial Sequence
  tag sequence for base balance
tatctcgcta

SEQ ID NO: 696 - DNA, 10 bp, Artificial Sequence
  tag sequence for base balance
gcgatggaag

SEQ ID NO: 697 - DNA, 10 bp, Artificial Sequence
  tag sequence for base balance
taactcgtgg

SEQ ID NO: 698 - DNA, 10 bp, Artificial Sequence
  tag sequence for base balance
acaaccagta

SEQ ID NO: 699 - DNA, 10 bp, Artificial Sequence
  tag sequence for base balance
agcgtacgtc

SEQ ID NO: 700 - DNA, 10 bp, Artificial Sequence
  tag sequence for base balance
tcgactctgt

SEQ ID NO: 701 - DNA, 10 bp, Artificial Sequence
  tag sequence for base balance
ctgtatgcca

SEQ ID NO: 702 - DNA, 10 bp, Artificial Sequence
  tag sequence for base balance
gacgagacct

SEQ ID NO: 703 - DNA, 10 bp, Artificial Sequence
  tag sequence for base balance
cgttggtaac

SEQ ID NO: 704 - DNA, 10 bp, Artificial Sequence
  tag sequence for base balance
gttcgataag

SEQ ID NO: 705 - DNA, 10 bp, Artificial Sequence
  tag sequence for base balance
caagcgcgat

SEQ ID NO: 706 - DNA, 10 bp, Artificial Sequence
  tag sequence for base balance
gtgtgagtgc

SEQ ID NO: 707 - DNA, 10 bp, Artificial Sequence
  tag sequence for base balance
gcagcataat

SEQ ID NO: 708 - DNA, 10 bp, Artificial Sequence
  tag sequence for base balance
ttcttgtgca

SEQ ID NO: 709 - DNA, 10 bp, Artificial Sequence
  tag sequence for base balance
tggcacattg

SEQ ID NO: 710 - DNA, 10 bp, Artificial Sequence
  tag sequence for base balance
aacaatacgc

SEQ ID NO: 711 - DNA, 10 bp, Artificial Sequence
  tag sequence for base balance
cgtattgctg

SEQ ID NO: 712 - DNA, 10 bp, Artificial Sequence
  tag sequence for base balance
actcgccaca

SEQ ID NO: 713 - DNA, 10 bp, Artificial Sequence
  tag sequence for base balance
gctgcttggt

SEQ ID NO: 714 - DNA, 10 bp, Artificial Sequence
  tag sequence for base balance
ttgtggatac

SEQ ID NO: 715 - DNA, 10 bp, Artificial Sequence
  tag sequence for base balance
accaaggcga

SEQ ID NO: 716 - DNA, 10 bp, Artificial Sequence
  tag sequence for base balance
agaagaccta

SEQ ID NO: 717 - DNA, 10 bp, Artificial Sequence
  tag sequence for base balance
tagctctgtc

SEQ ID NO: 718 - DNA, 10 bp, Artificial Sequence
  tag sequence for base balance
cgacttctat

SEQ ID NO: 719 - DNA, 10 bp, Artificial Sequence
  tag sequence for base balance
ctctccaacg

SEQ ID NO: 720 - DNA, 10 bp, Artificial Sequence
  tag sequence for base balance
gatgaagacg

SEQ ID NO: 721 - DNA, 10 bp, Artificial Sequence
  tag sequence for base balance
tccagctcat

SEQ ID NO: 722 - DNA, 10 bp, Artificial Sequence
  tag sequence for base balance
accaagactc

SEQ ID NO: 723 - DNA, 10 bp, Artificial Sequence
  tag sequence for base balance
caactgcgca

SEQ ID NO: 724 - DNA, 10 bp, Artificial Sequence
  tag sequence for base balance
agtggatatc

SEQ ID NO: 725 - DNA, 10 bp, Artificial Sequence
  tag sequence for base balance
gtactaagag

SEQ ID NO: 726 - DNA, 10 bp, Artificial Sequence
  tag sequence for base balance
gtgtacgtcg

SEQ ID NO: 727 - DNA, 10 bp, Artificial Sequence
  tag sequence for base balance
cggtctctgt

SEQ ID NO: 728 - DNA, 10 bp, Artificial Sequence
  tag sequence for base balance
tatgctgaga

SEQ ID NO: 729 - DNA, 10 bp, Artificial Sequence
  tag sequence for base balance
aataggtagg

SEQ ID NO: 730 - DNA, 10 bp, Artificial Sequence
  tag sequence for base balance
agcttgcgct

SEQ ID NO: 731 - DNA, 10 bp, Artificial Sequence
  tag sequence for base balance
tggagccgat

SEQ ID NO: 732 - DNA, 10 bp, Artificial Sequence
  tag sequence for base balance
ccgcatacta

SEQ ID NO: 733 - DNA, 10 bp, Artificial Sequence
  tag sequence for base balance
ttcgatgacg

SEQ ID NO: 734 - DNA, 10 bp, Artificial Sequence
  tag sequence for base balance
gatccaatac

SEQ ID NO: 735 - DNA, 10 bp, Artificial Sequence
  tag sequence for base balance
gcattctcgc

SEQ ID NO: 736 - DNA, 10 bp, Artificial Sequence
  tag sequence for base balance
ctagcagtta

SEQ ID NO: 737 - DNA, 10 bp, Artificial Sequence
  tag sequence for base balance
cgttgacgct

SEQ ID NO: 738 - DNA, 10 bp, Artificial Sequence
  tag sequence for base balance
agtatatgcg

SEQ ID NO: 739 - DNA, 10 bp, Artificial Sequence
  tag sequence for base balance
acctccgcta

SEQ ID NO: 740 - DNA, 10 bp, Artificial Sequence
  tag sequence for base balance
caacacgaat

SEQ ID NO: 741 - DNA, 10 bp, Artificial Sequence
  tag sequence for base balance
ttgagtaagg

SEQ ID NO: 742 - DNA, 10 bp, Artificial Sequence
  tag sequence for base balance
gaagctctta

SEQ ID NO: 743 - DNA, 10 bp, Artificial Sequence
  tag sequence for base balance
tccgagatgc

SEQ ID NO: 744 - DNA, 10 bp, Artificial Sequence
  tag sequence for base balance
gtgctgtcac

SEQ ID NO: 745 - DNA, 10 bp, Artificial Sequence
  tag sequence for base balance
agcttccagc

SEQ ID NO: 746 - DNA, 10 bp, Artificial Sequence
  tag sequence for base balance
tgcctatcgc

SEQ ID NO: 747 - DNA, 10 bp, Artificial Sequence
  tag sequence for base balance
caatctcgcg

SEQ ID NO: 748 - DNA, 10 bp, Artificial Sequence
  tag sequence for base balance
cttcctacaa

SEQ ID NO: 749 - DNA, 10 bp, Artificial Sequence
  tag sequence for base balance
acgaggtact

SEQ ID NO: 750 - DNA, 10 bp, Artificial Sequence
  tag sequence for base balance
gatgaaggat

SEQ ID NO: 751 - DNA, 10 bp, Artificial Sequence
  tag sequence for base balance
tcgagcgtta

SEQ ID NO: 752 - DNA, 10 bp, Artificial Sequence
  tag sequence for base balance
gtagagattg

SEQ ID NO: 753 - DNA, 10 bp, Artificial Sequence
  tag sequence for base balance
acggctagag

SEQ ID NO: 754 - DNA, 10 bp, Artificial Sequence
  tag sequence for base balance
gctatagctt

SEQ ID NO: 755 - DNA, 10 bp, Artificial Sequence
  tag sequence for base balance
tgcgtcatgg

SEQ ID NO: 756 - DNA, 10 bp, Artificial Sequence
  tag sequence for base balance
ttccaacatc

SEQ ID NO: 757 - DNA, 10 bp, Artificial Sequence
  tag sequence for base balance
cggtgttgga

SEQ ID NO: 758 - DNA, 10 bp, Artificial Sequence
  tag sequence for base balance
gattgcgcct

SEQ ID NO: 759 - DNA, 10 bp, Artificial Sequence
  tag sequence for base balance
ataacgctca

SEQ ID NO: 760 - DNA, 10 bp, Artificial Sequence
  tag sequence for base balance
caacagtaac

SEQ ID NO: 761 - DNA, 10 bp, Artificial Sequence
  tag sequence for base balance
actggaggag

SEQ ID NO: 762 - DNA, 10 bp, Artificial Sequence
  tag sequence for base balance
gccggtaagt

SEQ ID NO: 763 - DNA, 10 bp, Artificial Sequence
  tag sequence for base balance
ctcacactta

SEQ ID NO: 764 - DNA, 10 bp, Artificial Sequence
  tag sequence for base balance
agacagtggc

SEQ ID NO: 765 - DNA, 10 bp, Artificial Sequence
  tag sequence for base balance
ctgatgtcct

SEQ ID NO: 766 - DNA, 10 bp, Artificial Sequence
  tag sequence for base balance
gagttcacaa

SEQ ID NO: 767 - DNA, 10 bp, Artificial Sequence
  tag sequence for base balance
tgttctcttc

SEQ ID NO: 768 - DNA, 10 bp, Artificial Sequence
  tag sequence for base balance
taacacgacg

SEQ ID NO: 769 - DNA, 31 bp, Artificial Sequence
  tag sequence for base balance
  misc_feature (1)..(31): solution 1 adapter 1
  misc_feature (1)..(1): 5phosphorylation
agtcggaggccaagcggtcttaggaagacaa

SEQ ID NO: 770 - DNA, 40 bp, Artificial Sequence
  tag sequence for base balance
  misc_feature (1)..(40): solution 1 adapter 2
  misc_feature (40)..(40): 3′-OH phosphorothioate
ttgtcttcctaacaggaacgacatggctacgatccgactt

SEQ ID NO: 771 - DNA, 64 bp, Artificial Sequence
  tag sequence for base balance
  misc_feature (1)..(64): solution 1 primer1
  misc_feature (1)..(1): 5′phosphorylation
  misc_feature (21)..(30): n is a,c,t org
ctctcagtacgtcagcagttnnnnnnnnnncaactccttggctcacagaacgacatggctacga

SEQ ID NO: 772 - DNA, 52 bp, Artificial Sequence
  tag sequence for base balance
  misc_feature (1)..(52): solution 1 primer2
  misc_feature (19)..(28): n is a, t, c or g
gcatggcgaccttatcagnnnnnnnnnnttgtcttcctaagaccgcttggcc

SEQ ID NO: 773 - DNA, 35 bp, Artificial Sequence
  tag sequence for base balance
  misc_feature (1)..(35): solution 2 adapter 1
  misc_feature (1)..(1): 5′phosphorylation
agtcggaggccaagcggtcttaggaagacaatcag

SEQ ID NO: 774 - DNA, 59 bp, Artificial Sequence
  tag sequence for base balance
  misc_feature (1)..(59): solution 2 adapter 2
ctgattgtcttcctaagcaactccttggctcacagaacgacatggctacgatccgactt

SEQ ID NO: 775 - DNA, 51 bp, Artificial Sequence
  tag sequence for base balance
  misc_feature (1)..(51): solution 2 primer1
  misc_feature (1)..(1): 5′phosphorylation
  misc_feature (21)..(30): n is a, t, c or g
ctctcagtacgtcagcagttnnnnnnnnnncaactccttggctcacagaac

SEQ ID NO: 776 - DNA, 50 bp, Artificial Sequence
  tag sequence for base balance
  misc_feature (1)..(50): solution 2 primer2
  misc_feature (19)..(28): n is a, t, c or g
gcatggcgaccttatcagnnnnnnnnnnttgtcttcctaagaccgcttgg

SEQ ID NO: 777 - DNA, 10 bp, Artificial Sequence
  tag sequence for base balance
  misc_feature (1)..(10): MGI001tag1
atgcatctaa

SEQ ID NO: 778 - DNA, 10 bp, Artificial Sequence
  tag sequence for base balance
  misc_feature (1)..(10): MGI002tag1
agctctggac

SEQ ID NO: 779 - DNA, 10 bp, Artificial Sequence
  tag sequence for base balance
  misc_feature (1)..(10): MGI003tag1
ctatcacgtg

SEQ ID NO: 780 - DNA, 10 bp, Artificial Sequence
  tag sequence for base balance
  misc_feature (1)..(10): MGI004tag1
ggactagtgg

SEQ ID NO: 781 - DNA, 10 bp, Artificial Sequence
  tag sequence for base balance
  misc_feature (1)..(10): MGI005tag1
gccaagtcca

SEQ ID NO: 782 - DNA, 10 bp, Artificial Sequence
  tag sequence for base balance
  misc_feature (1)..(10): MGI006tag1
cctgtcaagc

SEQ ID NO: 783 - DNA, 10 bp, Artificial Sequence
  tag sequence for base balance
  misc_feature (1)..(10): MGI007tag1
tagaggtctt

SEQ ID NO: 784 - DNA, 10 bp, Artificial Sequence
  tag sequence for base balance
  misc_feature (1)..(10): MGI008tag1
tatggcaact

SEQ ID NO: 785 - DNA, 10 bp, Artificial Sequence
  tag sequence for base balance
  misc_feature (1)..(10): MGI001tag2
tagaggacaa

SEQ ID NO: 786 - DNA, 10 bp, Artificial Sequence
  tag sequence for base balance
  misc_feature (1)..(10): MGI002tag2
cctagcgaat

SEQ ID NO: 787 - DNA, 10 bp, Artificial Sequence
  tag sequence for base balance
  misc_feature (1)..(10): MGI003tag2
gtagtcatcg

SEQ ID NO: 788 - DNA, 10 bp, Artificial Sequence
  tag sequence for base balance
  misc_feature (1)..(10): MGI004tag2
gctgagctgt

SEQ ID NO: 789 - DNA, 10 bp, Artificial Sequence
  tag sequence for base balance
  misc_feature (1)..(10): MGI005tag2
aacctagata

SEQ ID NO: 790 - DNA, 10 bp, Artificial Sequence
  tag sequence for base balance
  misc_feature (1)..(10): MGI006tag2
ttgccatctc

SEQ ID NO: 791 - DNA, 10 bp, Artificial Sequence
  tag sequence for base balance
  misc_feature (1)..(10): MGI007tag2
agatcttgcg

SEQ ID NO: 792 - DNA, 10 bp, Artificial Sequence
  tag sequence for base balance
  misc_feature (1)..(10): MGI008tag2
cgctatcggc

SEQ ID NO: 793 - DNA, 20 bp, Artificial Sequence
  tag sequence for base balance
  misc_feature (1)..(20): universial amplification sequence
  misc_feature (1)..(1): 5′
ctctcagtacgtcagcagtt

SEQ ID NO: 794 - DNA, 34 bp, Artificial Sequence
  tag sequence for base balance
  misc_feature (1)..(34): universial primer
caactccttggctcacagaacgacatggctacga

SEQ ID NO: 795 - DNA, 18 bp, Artificial Sequence
  tag sequence for base balance
  misc_feature (1)..(18): universial primer
gcatggcgaccttatcag

SEQ ID NO: 796 - DNA, 24 bp, Artificial Sequence
  tag sequence for base balance
  misc_feature (1)..(24): universial primer
ttgtcttcctaagaccgcttggcc

SEQ ID NO: 797 - DNA, 21 bp, Artificial Sequence
  tag sequence for base balance
  misc_feature (1)..(21): universial amplification sequence
caactccttggctcacagaac

SEQ ID NO: 798 - DNA, 22 bp, Artificial Sequence
  tag sequence for base balance
  misc_feature (1)..(22): universial amplification sequence
ttgtcttcctaagaccgcttgg

(19) United States
(12) Patent Application Publication
(10) Pub. No.: US 2024/0271123 A1
(43) Pub. Date: Aug. 15, 2024

(54) Double-End Library Tags Composition And Application Thereof In MGI Sequencing Platform

(71) Applicant: Nanodigmbio (Nanjing) Biotechnology Co., LTD, Nanjing,Jiangsu (CN)

(72) Inventors: Biao WANG, Nanjing, Jiangsu (CN); Yugang HU, Nanjing,Jiangsu (CN); Qiang WU, Nanjing,Jiangsu (CN)

(21) Appl. No.: 18/013,578

(22) PCT Filed: Dec. 28, 2020
(86) PCT No.: PCT/CN2020/139919

§ 371 Date: Dec. 29, 2022

(30) Foreign Application Priority Data
Aug. 19, 2020 (CN) 202010838955.X

(51) Int. Cl. C12N 15/10

(52) U.S. Cl. CPC C12N 15/1065; C12N 15/1068

(57) ABSTRACT

The invention provides a double-end library tags composition and application thereof in MGI sequencing platform. The double-end library tags composition includes a plurality of 5′-end library tags and a plurality of 3′-end library tags, the lengths of the plurality of 5′-end library tags are all the same, the lengths of the plurality of 3′-end library tags are all the same, and in the double-end library tags composition, the occurrences of each base at the same position are also all the same.

### CROSS-REFERENCE TO RELATED APPLICATION

The present application is a National Stage of International Patent Application No. PCT/CN2020/139919, filed on Dec. 28, 2020, and claims priority to and interest of patent application No. 202010838955.X, filed to the China National Intellectual Property Administration on Aug. 19, 2020, the disclosures of which are hereby incorporated by reference in its entirety.

### SEQUENCE LISTING

The instant application contains a Sequence Listing which has submitted electronically in ASCII format and is hereby incorporated by reference in its entirety. Said ASCII copy is named PN193247 SEQ LIST.txt and is 166,000 bytes in size. The sequence listing contains 798 sequences, which is identical in substance to the sequences disclosed in the PCT application, and only the translated title has been amended, and includes no new matter.

### TECHNICAL FIELD

The invention relates to the field of plasma DNA library construction, more specifically, refers to a double-end library tags composition and application thereof in the MGI sequencing platform.

### BACKGROUND

In the sequencing process of the MGI high-throughput sequencer, in order to realize more samples sequencing, each sample needs to be labeled with a different index and sequenced and then the data is split through bioinformatic analysis depending on the indexes information. However, at present single-end library tags are basically used in MGI sequencing platform. As single-end library tags (index) have natural defects, it is easy to cause data crosstalk problems between different samples. Due to the contamination of adapters or primers in synthesis, experimental process and sequencing, crosstalk problems are inevitable. Therefore, it is necessary to solve the low-frequency mutual crosstalk problems between different samples. The best way is to use double-end library tags, which can effectively remove the mutual crosstalk problems between different samples.

However, compared with single-end library tags, applying double-end library tags, whether the sequencer can accurately read the double-end library tags or not, will seriously affect the effective splitting of the sequencing data through bioinformatic analysis. If there is a problem with reading the sequences of the double-end library tags, the sequencing data splitting rate will be reduced, thereby increasing the sequencing cost.

Therefore, how to use double-end tags to label pooled libraries, which can not only reduce the sample crosstalk problems but also improve the sequencing data splitting rate, is a problem to be solved.

### SUMMARY

The main purpose of the invention is to provide a double-end library tags composition and application thereof in MGI sequencing platform, to solve the sample crosstalk problems when using the single-end library tags in MGI sequencing platform.

In order to achieve the purpose, according to an aspect of the invention, the invention provides a double-end library tags composition, and the double-end library tags composition includes a plurality of 5′ end library tags and a plurality of 3′ end library tags, the lengths of the 5′ end library tags are all the same, the lengths of the 3′ end library tags are all the same, and the occurrences of each base at the same position are also all the same.

Further, the lengths of the 5′ end library tags are all the same with the lengths of the 3′ end library tags, preferably, are any fixed lengths between 6˜10 bp; preferably, in the double-end library tags composition, there are at least 3 base differences between any two library tags, and the number of continuous same bases in any library tag does not exceed 3, GC contents in all library tags are all 40-60%. preferably, the double-end library tags composition comprises a combination of 4-balanced double-end library tags, or a combination of 8-balanced double-end library tags, wherein the combination of 4-balanced double-end library tags comprises 4n 5′ end library tags and 4n 3′ end library tags, and the combination of 8-balanced double-end library tags comprises 8n 5′ end library tags and 8n 3′ end library tags, wherein n is an integer greater than or equal to 1.

Further, in the combination of 4-balanced double-end library tags, the 5′ end library tags are selected from any one or more of the 96 groups shown in Table 1, and the 3′ end library tags are selected from any one or more of the 96 groups shown in Table 1 that are different from the 5′-end library tags.

Further, in the combination of 8-balanced double-end library tags. the 5′ end library tags are selected from any one or more of the 48 groups shown in Table 2, and the 3′ end library tags are selected from any one or more of the 48 groups shown in Table 2 that are different from the 5′-end library tags.

According to the second aspect of the invention, the invention provides composition of amplification primers with double-end library tags based on MGI sequencing platform, and the composition of amplification primers includes a plurality of amplification primer pairs with double-end library tags, each amplification primer pair comprises a 5′ end library tag and a 3′ end library tag, and the lengths of multiple 5′ end library tags of the amplification primer pairs are all the same, and the lengths of multiple 3′ end library tags of the amplification primer pairs are all the same, and the occurrences of each base at the same position are also all the same.

Further, the lengths of multiple 5′ end library tags of the amplification primer pairs are all the same with the lengths of multiple 3′ end library tags of the amplification primer pairs; preferably, the lengths of the multiple 5′ end library tags and the lengths of the multiple 3′ end library tags are any fixed lengths between 6 ˜10bp; preferably, in the composition, there are at least 3 base differences between any two library tags, and the number of continuous same bases in any library tag does not exceed 3; preferably, GC contents in all library tags are all 40-60%; preferably, the composition comprises a combination of 4n 4-balanced amplification primer pairs, or a combination of 8n 8-balanced amplification primer pairs, wherein n is an integer greater than or equal to 1.

Further, in the combination of 4n 4-balanced amplification primer pairs, the 5′ end library tags are selected from any one or more of the 96 groups shown in Table 1, and the 3′ end library tags are selected from any one or more of the 96 groups shown in Table 1 that are different from the 5′-end library tags; preferably, in the combination of 8n 8-balanced amplification primer pairs, the 5′ end library tags are selected from any one or more of the 48 groups shown in Table 2, and the 3′ end library tags are selected from any one or more of the 48 groups shown in Table 2 that are different from the 5′-end library tags.

Further, each amplification primer pair further comprises a 5′ end universal amplification sequence and a 3′ end universal amplification sequence, the 5′ end universal amplification sequence comprises an universal upstream sequence of the 5′ end library tag and an universal downstream sequence of the 5′ end library tag. and the 3′ end universal amplification sequence comprises an universal upstream sequence of the 3′ end library tag and an universal downstream sequence of the 3′ end library tag; preferably, the universal upstream sequence of the 5′ end library tag is SEQ ID NO: 793, and the universal downstream sequence of the 5′ end library tags is SEQ ID NO: 794; the universal upstream sequence of the 3′ end library tag is SEQ ID NO: 795, and the universal downstream sequence of the 3′ end library tag is SEQ ID NO: 796; or
the universal upstream sequence of the 5′ end library tag is SEQ ID NO: 793, and the universal downstream sequence of the 5′ end library tag is SEQ ID NO: 797; the universal upstream sequence of the 3′ end library tag is SEQ ID NO: 795, and the universal downstream sequence of the 3′ end library tag is SEQ ID NO: 798.

According to the third aspect of the invention, the invention also provides a sequencing library construction kit, which includes any one of the above composition of amplification primers.

Further, the kit further comprises bubble adapters, wherein the bubble adapters comprise a first adapter sequence and a second adapter sequence, the first adapter sequence is SEQ ID NO: 769, and the second adapter sequence is SEQ ID NO: 770, or the first adapter sequence is SEQ ID NO: 773, and the second adapter sequence is SEQ ID NO: 774.

According to the fourth aspect of the invention, the invention provides a method for constructing a sequencing library based on MGI sequencing platform, comprising applying any one of the kit to construct.

According to the fifth aspect of the invention, the invention provides a sequencing library including the above double-end library tags combination, or any one of the above combinations of amplification primers.

By introducing the double-end library tags and the optimized double-end library tags combination, when applying the double-end library tags for sequencing data splitting, the crosstalk problems caused by synthesis, experimental process and machine sequencing can be solved, and the results will be more accurate. Further, by controlling that the lengths of 5′ end library tags and the lengths of the 3′ end library tags are the same, and limiting the occurrences of each base at the same position are the same, the bases of the double-end tags in the composition have the same occurrence, so when the adapters or library amplification primers with the double-end tags of the composition are synthesized, multiple libraries with good base-balanced double-end tags can be obtained. When these multiple libraries are pooled and sequenced on the machine, the sequences of the double-end tags can be read accurately and the sequencing data can be split effectively.

### BRIEF DESCRIPTION OF THE DRAWINGS

The accompanying drawings, which form a part of this application, are provided to further understand the present invention, the illustrative embodiments of the present invention and the description thereof are intended to explain the present invention and are not intended to limit thereto. In the drawings:

FIG. 1A, FIG. 1B, and FIG. 1C show the advantages of MGI sequencing platform using double-end tags over single-end tags to remove crosstalk problems;

FIGS. 2A and 2B show two forms of MGI single-end tag adapter;

FIGS. 3A and 3B show two forms of MGI double-end tag adapter;

FIG. 4 shows the process of constructing a library using two double-end tags based on MGI platform;

FIG. 5 shows that the inventions applying the double-end tags of the present invention are compatible with the inventions applying the single-end tags;

FIG. 6 shows an adapter in which the double-end tags amplification primers and the single-end tags amplification primers are compatible;

FIGS. 7A and 7B show the base-balanced type of 4-balanced and 8-balanced sequences;

FIG. 8 shows the comparison of base-balance between 4-balanced and 8-balanced tags in the hybrid process;

FIG. 9 shows the output comparison of the two library construction methods;

FIG. 10 shows the difference in sequencing data split between 4-balanced and 8-balanced tags in 12 pooled samples sequencing processes.

### DETAILED DESCRIPTION OF THE EMBODIMENTS

It should be noted that the embodiments in the present application and the features in the embodiments may be combined with each other without conflict. The present invention will be described in detail below with reference to the embodiments.

Interpretation of specific terms:

Double-end tag adapters: For high-throughput sequencing, a universal sequencing adapter is required to connect to the ends of each fragment. Each non-complementary region of the adapter has a variable sequence that is a tag sequence, which is used to split data during sequencing.

Base balance of tag sequences: DNA sequence consists of four bases, namely A, T. G and C. For effective reading during sequencing, a set of tag sequences is combined to ensure the base ratio of each position in the tag sequence is equal.

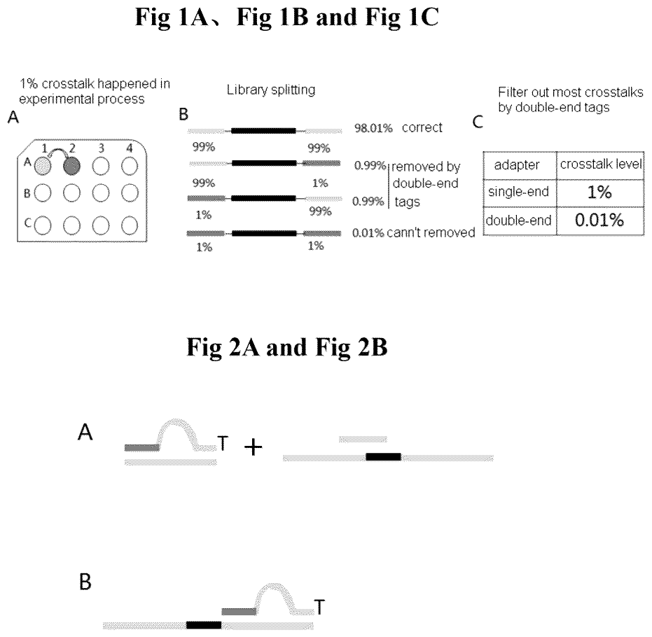

As mentioned in the background, when single-end tags are used to construct libraries for MGI high-throughput sequencing, there are some crosstalk problems between samples (this is a phenomenon that also exists in Illumina sequencing platform. Although MGI platform is much different from the Illumina platform, the process of adapter sequence synthesis, library construction, and hybridization capture inevitably causes crosstalk problems between samples). As shown in FIG. 1A, if there are 1% mutual crosstalk problems in the experimental process, whether it is in adapter synthesis, library construction, hybridization capture, or machine sequencing, there will be the same crosstalk problems. The best way to solve the crosstalk problems between samples is to introduce the double-end tags in the process of library construction. As shown in FIG. 1B, the crosstalk problems can only be solved by introducing the double-end tags meanwhile controlling experimental processes as much as possible. As shown in FIG. 1C, the double-end tags will reduce crosstalk problems by 100 times (1% to 0.01%) than the single-end tag.

In order to solve the sample crosstalk problems in MGI sequencing platform, this invention also tries to change the single-end tags to the double-end tags. The research and development ideas and process are as follows.

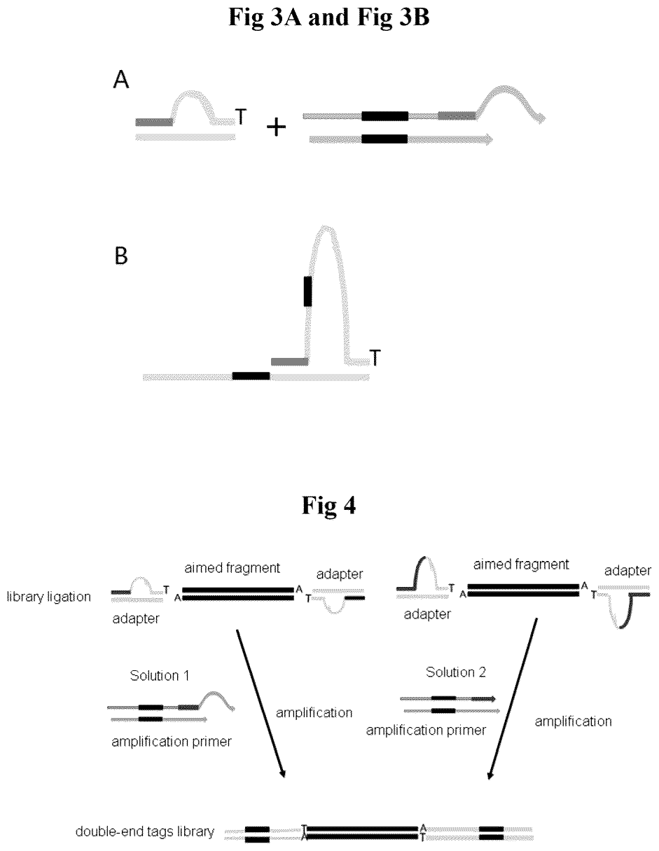

Bubble adapters are used in MGI library construction. Unlike Illumina Y-type adapters, MGI single-end tags can be fused into the adapters (as shown in FIG. 2B) or separately used (FIG. 2A): while the double-end tag sequences cannot be fused with the front end sequence (as shown in FIG. 3B, if the tag sequence is fused at the front end, since the front end region is only 7 bp, the vesicle structure will be longer, and the stability of this structure is extremely poor, and the efficiency is very low. And the implementation effect is not as efficient as the truncated structure where the tag sequence primers and the universal adapter are separated). And the universal adapter and the double-end tag amplification primers can be separately used (as shown in FIG. 3A). The double-end tags were connected according to the structure shown in FIG. 3A, and inventors found the large vesicle in the middle of the bubble adapter would affect the stability of the annealing secondary structure, and affect the ligation of the adapters (average efficiency is 20%-40%). MGI bubble adapter is different from Illumina Y-adapter in which the double-end tags can be fused together.

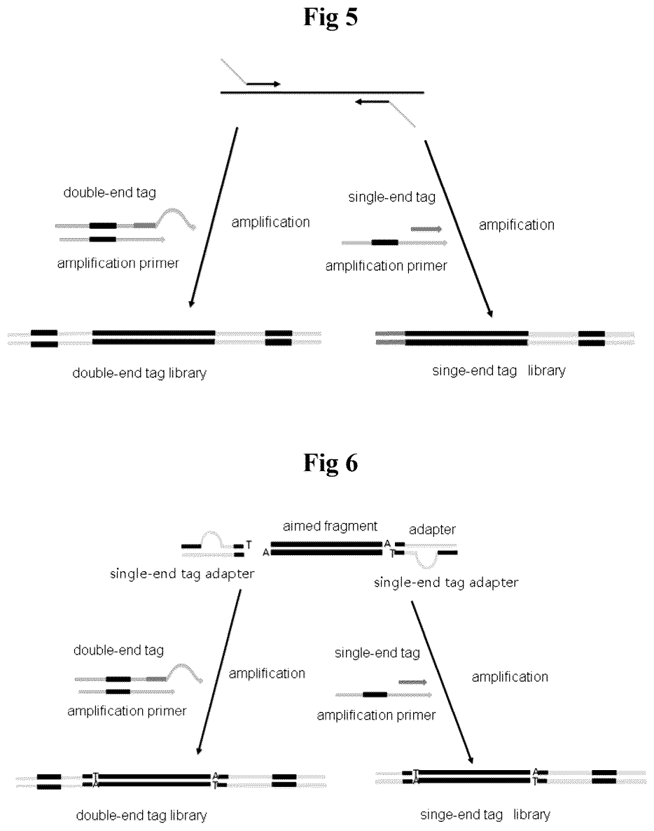

Further research found that when the unpaired bases in the middle region of the MGI bubble adapters can be 30±5bp, and the paired base is 20±2bp, it is easier to form a stable annealing ligation, improving the ligation efficiency, as shown in the Solution 1 of FIG. 4. When the unpaired bases in the middle region can also be 45±5bp, and the paired base is 25±2bp, it is easier to form a stable annealing ligation, improving the ligation efficiency, as shown in the Solution 2 of FIG. 4. The inventorss further found that compared with the Solution 2, the Solution 1 has the following advantages: first, when the vesicle region is 30±5bp, adapters anneal stably, the region to be complementary with is short and the stability is benefit for ligation. Second, being compatible with amplicons with single-end tags, and the amplicons can be switched between single-end tags and double-end tags, as shown in FIG. 5. It is compatible with single-end tag adapters, as shown in FIG. 6.

The inventors further found that although Solution 2 has many advantages over the Solution 1, both two solutions can work if you want to obtain the sequencing library in MGI sequencing platform with double-end tags. If the constructed library with double-end tags is used for machine sequencing and the sequencing data is split after sequencing, the inventors found that the base balance requirements of MGI double-end tag adapters during sequencing are more stringent than that of the single-end tag adapters, and the sequencing data can only be split when the tag sequences at two ends are both correct, as shown in FIG. 1B. That is, although the double-end tags solve the crosstalk problems between samples, the base balance requirements for machine sequencing are extremely stringent, and the poor base balance will seriously affect the accurate reading of the sequencing data, which in turn affects the effective sequencing split.

In order to split the sequencing data more accurately, taking the base number of the double-end tags are both 10 as an example, the inventors have optimized the base balance of the double-end tags according to the following rules, and the rules for base screening are as follows: 1) There are 3 base differences between each tag sequence; 2) The GC content of each sequence is 0.4-0.6; 3) The number of continuous same bases cannot exceed 3. According to these rules, the secondary structure of each selected tag sequence was evaluated to see whether a secondary structure such as hairpin folds is formed between the tag sequence and the universal primer at the 3′ end of the amplification primer, which will reduce the amplification efficiency, affects the balance of each tag base in the pooled sample libraries, further affects the reading accuracy of tag sequence, and therefore reduces the accuracy of sequencing data splitting.

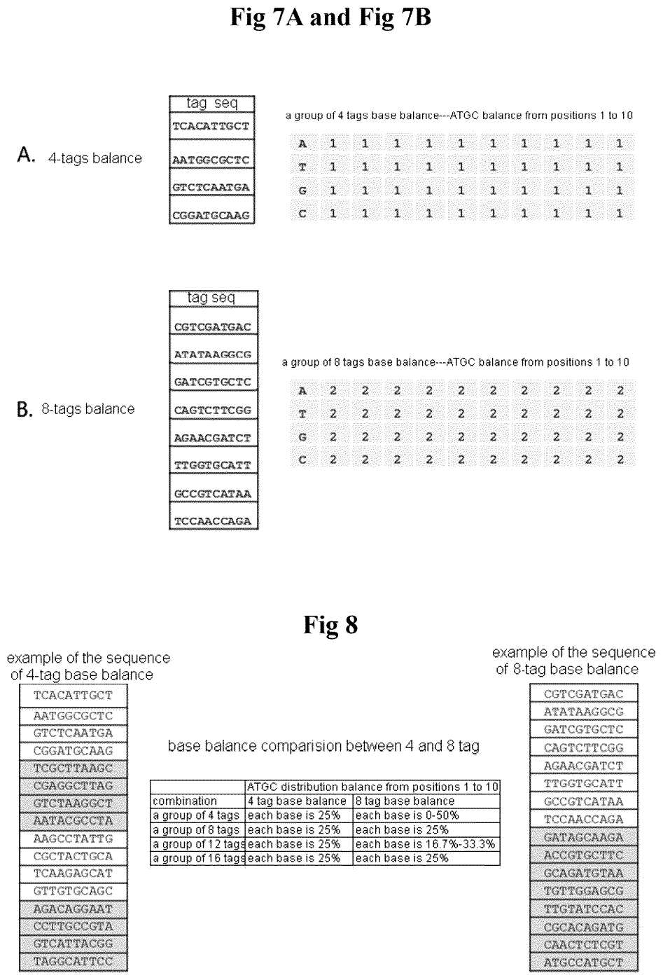

According to the above optimized screening rules, the present invention optimizes 384 types of 4-balanced tags and 384 types of 8-balanced tags sequences. 4-balanced tags refer to a group of 4 tags sequences, as shown in FIG. 7A (first 1-4 tags shown in Table 4). A group of 4 tags sequences refers to base A, T, G, or C occurs once in each position from the 1^{st} to the 10^{th} position of each tag. Similarly, the 8-balanced tags refer to a group of 8 tags sequences, as shown in FIG. 7B (first 1-8 tags shown in Table 5). A group of 8 tags sequences refers to base of A, T, G, or C occurs twice in each position from the 1^{st} to the 10^{th} position of each tag.

According to multiple tests of the invention, the group of 4-balanced tags is the smallest unit of balance and the best combination. 4-balanced tags combinations can be combined into 4, 8. 12, and 16 combinations that are 4 fold-balanced, and 8-balanced tags combinations need to be combined into 8 and 16 combinations that are 8 fold-balanced. As shown in FIG. 8 (the tag sequence of the 4-balanced tags combination on the left corresponds to the library tag combination carried by the first 4 sets of amplification primer sets in Table 1, and the library tag of the 8-balanced tags combination on the right corresponds to the library tag combination carried by the first two sets of amplification primer sets in Table 2), when the 4-balanced tags libraries are pooled and sequenced on the machine, the bases are balanced, and the proportion of each base is 25%. And when the 8-balanced tags combinations are used, the proportion of each base is 0-50%. When the 8 folds, for example, 8 or 16 samples are pooled on the machine, the proportion of each base after the library tags combination can be balanced, and each is 25%. When 12 samples are pooled and sequenced on the machine, the proportion of each base in the 8-balanced tags combination is between 16.7% and 33.3%.

In addition, the balance of non-integer fold of 4 tags is also better than the combination of 8-balanced tags, and the application of 4-balanced tags is more conducive. As the sequencing throughput of MGI sequencer becomes higher and higher, the optimized 384 types of 4-balanced tags combinations in the present application make the four close libraries with 4-balanced tags be sequenced effectively (see Table 1 for the 4-balanced tags combinations). The optimized 384 8-balanced tags combinations also make the eight close libraries with 8-balanced tags be sequenced effectively (see Table 2 for the 8-balanced tags combination).

Preferably, when the two balanced tags are used for forming the double-end amplification primers, the sequence of primer 1 is a forward arrangement of 384 numbers, and the primer 2 is a reverse arrangement of 384 numbers, which is a recommended arrangement of the present invention. In practical applications, it can also be combined and arranged according to actual needs. For example, as shown in Table 1, when primer 1 is selected in any of the 96 groups, primer 2 can be selected in any of the remaining 95 groups. Of course, if the number of samples to be pooled is greater than 4, such as 8 or 12, the number of the tag groups of the primer 1 just need to be different from that of the primer 2. For example, the primer 1 are selected from the first 3 groups, and primer 2 can be selected any 3 groups from the remaining 93 groups. As long as 4 fold samples are pooled and sequenced on the machine, the double-end library tags can be selected according to this rule.

When the number of the pooled samples is not integer fold of 4, the 4 samples with large amount of sequencing data shall be arranged in one set of balanced tag combinations, and the samples with small amount of sequencing data shall be arranged in another set of other balanced tag combinations. The 4-balanced tags combinations have obvious advantages over the 8-balanced tags combinations in this situation. 4-balanced tags combinations have an advantage over 8 balanced combinations for integer fold of 4 (4, 12, 20), and the combination of non-integer fold of 4 is also better than the 8-balanced tags combination, and the balance is better than that of the 8-balanced tags combination when the number of samples to be pooled is 4n+1 and 4n+2. Therefore, the 4-balanced tags combination has the following advantages: 1) The combinations of 4-balanced tags are twice as many as the 8-balanced ones; 2) For the three groups of unbalanced arrangements, the balance in the combinations of 4n+1 and 4n+2 groups is also better than the combination of 8-balanced tags; 3) When there is a difference in the amount of sequencing data between samples, the combinations of 4-balanced tags is better arranged close to the balance, and the samples for large amount of sequencing data are prioritized in the balanced combination, and it can be unbalanced for the samples for small amount of sequencing data.

TABLE 1
4-balanced group

| | SEQ | | | SEQ | | | SEQ | |
|---|---|---|---|---|---|---|---|---|
| Group | ID | | Group | ID | | Group | ID | |
| code | NO: | Sequence | code | NO: | Sequence | code | NO: | Sequence |
| 1 | 1 | tcacattgct | 33 | 129 | gcgaccttga | 65 | 257 | aatgactggt |
| | 2 | aatggcgctc | | 130 | atcgtgagtt | | 258 | gtgccacaac |
| | 3 | gtctcaatga | | 131 | cgtcgtcaac | | 259 | cgaatgatcg |
| | 4 | cggatgcaag | | 132 | taataagccg | | 260 | tcctgtgcta |
| 2 | 5 | tcgcttaagc | 34 | 133 | ccacttagta | 66 | 261 | tgtgaattgg |
| | 6 | cgaggcttag | | 134 | agtagctagt | | 262 | aaccggcctt |
| | 7 | gtctaaggct | | 135 | gacgaactcg | | 263 | ctatccgacc |
| | 8 | aatacgccta | | 136 | ttgtcggcac | | 264 | gcgattagaa |
| 3 | 9 | aagcctattg | 35 | 137 | agcatatcgt | 67 | 265 | cgtaaccgca |
| | 10 | cgctactgca | | 138 | gtagacggag | | 266 | gactgataac |
| | 11 | tcaagagcat | | 139 | cattctcatc | | 267 | atgcttactg |
| | 12 | gttgtgcagc | | 140 | tcgcggatca | | 268 | tcagcggtgt |
| 4 | 13 | agacaggaat | 36 | 141 | ctggaggcaa | 68 | 269 | acataacacc |
| | 14 | ccttgccgta | | 142 | gatacttgtg | | 270 | cacctgaggt |
| | 15 | gtcattacgg | | 143 | tgcctccact | | 271 | gtgactgtaa |
| | 16 | taggcattcc | | 144 | acatgaatgc | | 272 | tgtggctctg |
| 5 | 17 | catatcatcg | 37 | 145 | tcagcagagg | 69 | 273 | ctcagactct |
| | 18 | gcacaacaat | | 146 | ctgtgcatta | | 274 | acacatgcta |
| | 19 | ttgtcgtggc | | 147 | agtaatcgac | | 275 | tgtgcctaag |
| | 20 | agcggtgcta | | 148 | gacctgtcct | | 276 | gagttgaggc |
| 6 | 21 | agccagtagg | 38 | 149 | aagcggtgaa | 70 | 277 | agccgttctc |
| | 22 | gtaagtgtac | | 150 | ctatcacact | | 278 | ttgttggtct |
| | 23 | tagtcacgtt | | 151 | gccgttatgc | | 279 | caagacaaga |
| | 24 | cctgtcacca | | 152 | tgtaacgctg | | 280 | gctacacgag |
| 7 | 25 | atcgtggatg | 39 | 153 | gcgtgtaact | 71 | 281 | gtgacgcgat |
| | 26 | tggagatcga | | 154 | catgtaccac | | 282 | aacctctctg |
| | 27 | cctcacagat | | 155 | tgccactgta | | 283 | tcttgagaga |
| | 28 | gaatctctcc | | 156 | ataacggtgg | | 28/ | cgagatatcc |
| 8 | 29 | gcagactgac | 40 | 157 | cttgaaggtt | 72 | 285 | gaaggattca |
| | 30 | ctcattaacg | | 158 | gcctctatgg | | 286 | tcgcctggtt |
| | 31 | tggtgagctt | | 159 | tagatccacc | | 287 | agtaacacgg |
| | 32 | aatccgctga | | 160 | agacggtcaa | | 288 | ctcttgcaac |
| 9 | 33 | tcgcatcaac | 41 | 161 | gctggattaa | 73 | 289 | agagacttac |
| | 34 | agaacagtga | | 162 | taacaccggc | | 290 | cttatggccg |
| | 35 | catgtctcct | | 163 | atgactgccg | | 291 | gcctgacgtt |
| | 36 | gtctggagtg | | 164 | cgcttgaatt | | 292 | tagcctaaga |
| 10 | 37 | gaggtctgtg | 42 | 165 | ctatagcgag | 74 | 293 | aagcatatcc |
| | 38 | ctatagacgt | | 166 | aacgttgttc | | 294 | ctaatccgtt |
| | 39 | tgcagtgacc | | 167 | gctaccacgt | | 295 | gcttggtcga |
| | 40 | actccactaa | | 168 | tcgcgataca | | 296 | tgcgcagaag |
| 11 | 41 | gcgaagtagg | 43 | 169 | tccgccaatc | 75 | 297 | gatcctgata |
| | 42 | tgcctaacct | | 170 | caactagtgt | | 298 | tcaagcacgg |
| | 43 | aatggtctac | | 171 | agtaagccaa | | 299 | ctgtagcgct |
| | 44 | ctatccggta | | 172 | gtgtgttgcg | | 300 | agcgtattac |
| 12 | 45 | tacgcttcag | 44 | 173 | gatcagatgg | 76 | 301 | tgtctgattg |
| | 46 | cggagcatct | | 174 | ctcgtaggtc | | 302 | accagacggt |
| | 47 | gtactagatc | | 175 | tcgtgtccat | | 303 | gtgtctgacc |
| | 48 | acttagcgga | | 176 | agaacctaca | | 304 | caagactcaa |
| 13 | 49 | atcactccat | 45 | 177 | ccgcattcct | 77 | 305 | tcctccacag |
| | 50 | gatcgcagtg | | 178 | gttggacata | | 306 | agacgaggtc |
| | 51 | ccggaattcc | | 179 | tgatcgaggc | | 307 | ctggattact |
| | 52 | tgattggaga | | 180 | aacatcgtag | | 308 | gatatgctga |
| 14 | 53 | cacaaggtcg | 46 | 181 | gttgcgcgaa | 78 | 309 | ctggtcaagg |
| | 54 | tctcgcagga | | 182 | acaagtaagc | | 310 | gctccattcc |
| | 55 | gtggtatcat | | 183 | cgcttctcct | | 311 | tgatgtcgaa |
| | 56 | agatctcatc | | 184 | tagcaagttg | | 312 | aacaaggctt |
| 15 | 57 | gatggagatt | 47 | 185 | aggcctcttc | 79 | 313 | catctagaca |
| | 58 | ctcattctgc | | 186 | gtaatgtcgt | | 314 | gcggatacag |
| | 59 | tggcagacaa | | 187 | cactgcagag | | 315 | ttctgcttgt |
| | 60 | acatcctgcg | | 188 | tctgaagaca | | 316 | agaacgcgtc |
| 16 | 61 | acgtcgcaga | 48 | 189 | gctaaggata | 80 | 317 | aagacgaact |
| | 62 | tgtataggct | | 190 | cacggttggt | | 318 | ccagtactgc |
| | 63 | caacacattg | | 191 | tgaccaccag | | 319 | gttcgctgta |
| | 64 | gtcggttcac | | 192 | atgttcatcc | | 320 | tgctatgcag |
| 17 | 65 | agccataagc | 49 | 193 | agctactctg | 81 | 321 | tacacgcgca |
| | 66 | gtattccgag | | 194 | caacgtgagt | | 322 | aggtacgcag |
| | 67 | cataggttca | | 195 | tcgatgctaa | | 323 | gttcgattgt |
| | 68 | tcggcagctt | | 196 | gttgcaagcc | | 324 | ccagttaatc |
| 18 | 69 | gttcggtcct | 50 | 197 | cgacatgtgt | 82 | 325 | taagatcgga |
| | 70 | tggattgtag | | 198 | gatgcgcata | | 326 | agctcggctt |
| | 71 | ccagaacgtc | | 199 | tcgtgatcag | | 327 | gttcgataag |
| | 72 | aactccaaga | | 200 | atcatcagcc | | 328 | ccgatcatcc |
| 19 | 73 | cgtacactgg | 51 | 201 | tcaatggcgg | 83 | 329 | actggactca |
| | 74 | gcacacagca | | 202 | cagtaactct | | 330 | caatagaggc |
| | 75 | atggtgtatc | | 203 | gttgcctgac | | 331 | gtcacttaag |
| | 76 | tactgtgcat | | 204 | agccgtaata | | 332 | tggctcgctt |
| 20 | 77 | cggcaatcag | 52 | 205 | ctaataggct | 84 | 333 | cgcactatgg |
| | 78 | gtagttcgga | | 206 | tctcctccac | | 334 | gaggtacatt |
| | 79 | tacaggaact | | 207 | agctgcatga | | 335 | tcttagtgac |
| | 80 | acttccgttc | | 208 | gaggagtatg | | 336 | atacgcgcca |
| 21 | 81 | tgctccacga | 53 | 209 | accacgtagc | 85 | 337 | tgaggcatat |
| | 82 | aatcaaggtc | | 210 | gaatgcagta | | 338 | attctatggc |
| | 83 | gtaggtcaat | | 211 | ttggtagcct | | 339 | ccgtagcatg |
| | 84 | ccgatgttcg | | 212 | cgtcatctag | | 340 | gacactgcca |
| 22 | 85 | gacgtgtgca | 54 | 213 | tcaatgaggt | 86 | 341 | acggcattaa |
| | 86 | tcgccacttc | | 214 | agcgaagctg | | 342 | gtaagcgagg |
| | 87 | ataagcacgt | | 215 | ctgtcttaac | | 343 | cattatcgct |
| | 88 | cgttatgaag | | 216 | gatcgcctca | | 344 | tgcctgactc |
| 23 | 89 | aatagagcca | 55 | 217 | gttccgaatg | 87 | 345 | gactcatcca |
| | 90 | gtacctcgac | | 218 | tacgtacgca | | 346 | acgaacatac |
| | 91 | ccggtgattg | | 219 | cggagttcac | | 347 | ttacgtcggt |
| | 92 | tgctactagt | | 220 | acatacgtgt | | 348 | cgtgtggatg |
| 24 | 93 | gatccggact | 56 | 221 | ctgaagagat | 88 | 349 | cctaacattc |
| | 94 | ttaggcacaa | | 222 | gaagcctcca | | 350 | atcgcacgca |
| | 95 | agcattcttg | | 223 | tgtcttcatc | | 351 | gaacgttcgg |
| | 96 | ccgtaatggc | | 224 | acctgagtgg | | 352 | tggttggaat |
| 25 | 97 | gtatagctgc | 57 | 225 | gtacgtcctt | 89 | 353 | aacaagtgag |
| | 98 | cagacatctg | | 226 | aactaggtca | | 354 | gttgttgctc |
| | 99 | agcggtggat | | 227 | tgtgtcaggc | | 355 | tgacgcaact |
| | 100 | tctctcaaca | | 228 | ccgacataag | | 356 | ccgtcactga |
| 26 | 101 | catccactgt | 58 | 229 | tccacacgtc | 90 | 357 | tgttattccg |
| | 102 | gccgtgaaca | | 230 | cggcacatga | | 358 | ctactcaaga |
| | 103 | ataagcggag | | 231 | gtatgttcct | | 359 | acgagacgtc |
| | 104 | tggtattctc | | 232 | aatgtggaag | | 360 | gacgcggtat |
| 27 | 105 | acagttctca | 59 | 233 | agacagacgt | 91 | 361 | gatgacgtta |
| | 106 | cattgagagc | | 234 | ttctgtggag | | 362 | agccgatacc |
| | 107 | gtcacgactg | | 235 | caggtcttcc | | 363 | ttatctcgag |
| | 108 | tggcactgat | | 236 | gctacacata | | 364 | ccgatgacgt |
| 28 | 109 | aatgattcgc | 60 | 237 | ctagcgacac | 92 | 365 | gtgagttcgc |
| | 110 | cgcttaagta | | 238 | aggcattact | | 366 | acacaacatg |
| | 111 | ttgagcgacg | | 239 | gacatccgga | | 367 | cgtgcgatca |
| | 112 | gcaccgctat | | 240 | tcttgagttg | | 368 | tacttcggat |
| 29 | 113 | acgaacggat | 61 | 241 | acaacagaag | 93 | 369 | ctatcggtgt |
| | 114 | gaacggacta | | 242 | cgcttgtgga | | 370 | gacattcaag |
| | 115 | tgctctctgg | | 243 | gttggtctcc | | 371 | agtgacacca |
| | 116 | cttgtatacc | | 244 | tagcacactt | | 372 | tcgcgatgtc |
| 30 | 117 | caccagcaca | 62 | 245 | gcttgcaata | 94 | 373 | cgagtcagtc |
| | 118 | tgtctgtag | | 246 | tggacgtgct | | 374 | ttgccagtga |
| | 119 | agtatcactc | | 247 | aacgaaccgg | | 375 | aacaagcact |
| | 120 | gcaggatggt | | 248 | ctacttgtac | | 376 | gcttgttcag |
| 31 | 121 | ttatccacgt | 63 | 249 | cggtgagtga | 95 | 377 | tagtgatgtg |
| | 122 | acggttcgtc | | 250 | gcaatgcatt | | 378 | cgtgtgacat |
| | 123 | gaccagtaag | | 251 | ttccattcac | | 379 | atccaccacc |
| | 124 | cgtagagtca | | 252 | aatgccagcg | | 380 | gcaactgtga |
| 32 | 125 | acgttaaggt | 64 | 253 | tactcttctc | 96 | 381 | atccaccggt |
| | 126 | ctcagcttag | | 254 | aggaaggtaa | | 382 | ccgtcattac |
| | 127 | tgtgctccta | | 255 | gttggacggt | | 383 | tgagtggcta |
| | 128 | gaacaggacc | | 256 | ccactcaacg | | 384 | gatagtaacg |

TABLE 2
384 types of 8-balanced tag sequences

| | SEQ | | | SEQ | | | SEQ | |
|---|---|---|---|---|---|---|---|---|
| Group | ID | | Group | ID | | Group | ID | |
| code | NO: | Sequence | code | NO: | Sequence | code | NO | Sequence |
| 1 | 385 | cgtcgatgac | 17 | 513 | cgtcactatt | 33 | 641 | cagaacgtgg |
| | 386 | atataaggcg | | 514 | gcgatgcaga | | 642 | gttcttctgt |
| | 387 | gatcgtgctc | | 515 | cgtgtcctag | | 643 | cggtgaagtc |
| | 388 | cagtcttcgg | | 516 | gaagaatgga | | 644 | gtaagatgag |
| | 389 | agaacgatct | | 517 | atgtgtggct | | 645 | tgccatcaca |
| | 390 | ttggtgcatt | | 518 | tcctgtacac | | 646 | tcctcggata |
| | 391 | gccgtcataa | | 519 | ttaccgattc | | 647 | acagtgtcct |
| | 392 | tccaaccaga | | 520 | aacacagccg | | 648 | aatgccacac |
| 2 | 393 | gatagcaaga | 18 | 521 | tcataccaag | 34 | 649 | gaccactcga |
| | 394 | accgtgcttc | | 522 | gaagcttact | | 650 | atggacaaca |
| | 395 | gcagatgtaa | | 523 | gtcagtaggt | | 651 | aacctacggt |
| | 396 | tgttggagcg | | 524 | ctgtgcgtag | | 652 | tctaggattg |
| | 397 | ttgtatccac | | 525 | agtgaatgga | | 653 | cggattctcg |
| | 398 | cgcacagatg | | 526 | tccacggttc | | 654 | cgatcttctc |
| | 399 | caactctcgt | | 527 | aatctgcctc | | 655 | tcatcggaat |
| | 400 | atgccatgct | | 528 | cggctaacca | | 656 | gttggaggac |
| 3 | 401 | aggcagctta | 19 | 529 | gacatacagt | 35 | 657 | acacatgcta |
| | 402 | tagcctagcg | | 530 | agaggcctca | | 658 | ccttaggacg |
| | 403 | atcacgtgcg | | 531 | cctgatattg | | 659 | ctaaccaatc |
| | 404 | cgttatgcgc | | 532 | gtgctgaact | | 660 | aacgcacgag |
| | 405 | caaggatcga | | 533 | cgatggtcag | | 661 | gtcggttcac |
| | 406 | gttgtcgtat | | 534 | aatacagcgc | | 662 | tggctcatgt |
| | 407 | gcaatcaatc | | 535 | tcgtcctgac | | 663 | tggtggttgt |
| | 408 | tcctgacaat | | 536 | ttccatggta | | 664 | gatatacgca |
| 4 | 409 | aggtgcctta | 20 | 537 | cgttcgactg | 36 | 665 | cacgaacact |
| | 410 | aagaaccaag | | 538 | cgctacactc | | 666 | tgcgtgagca |
| | 411 | catacatgac | | 539 | atagatcggt | | 667 | gtactgacga |
| | 412 | tgcctggtga | | 540 | accagattca | | 668 | ccgtgagttc |
| | 413 | tctcggagtt | | 541 | gatacggagg | | 669 | acttacttgg |
| | 414 | gtcgtagact | | 542 | ttaggaggaa | | 670 | agaagctcat |
| | 415 | gcatataccg | | 543 | gcgcttctac | | 671 | gttactggac |
| | 416 | ctagcttcgc | | 544 | tagctctact | | 672 | tagcctcatg |
| 5 | 417 | gacgtatcaa | 21 | 545 | ccacctgctt | 37 | 673 | tgacaatgac |
| | 418 | cctgctagga | | 546 | cgtaggtctg | | 674 | aatcacctct |
| | 419 | caacttggcg | | 547 | tcttagtgac | | 675 | aacagacctg |
| | 420 | atgcgacctc | | 548 | atcctagaac | | 676 | gctagtgtgt |
| | 421 | gttaaggacg | | 549 | aggtgtaggt | | 677 | gcggttgata |
| | 422 | tccaagttgt | | 550 | gtaaccatca | | 678 | ctagcgacac |
| | 423 | aggtccattc | | 551 | taggtccaga | | 679 | cggttgaacg |
| | 424 | tgatgccaat | | 552 | gacgaactcg | | 680 | ttctcctgga |
| 6 | 425 | cctaagagtt | 22 | 553 | ccaacagatt | 38 | 681 | agttagctgg |
| | 426 | gatactagct | | 554 | cctctatctc | | 682 | agtcctgtaa |
| | 427 | aaggcatctc | | 555 | gagtgtctca | | 683 | taaggccggt |
| | 428 | tggcatctgg | | 556 | ttggcttaag | | 684 | gccttatcct |
| | 429 | acactggagc | | 557 | agtcgcatgg | | 685 | cagcattcaa |
| | 430 | gtatgccaca | | 558 | atcttgcggc | | 686 | tcgagcaatc |
| | 431 | ctcttagtag | | 559 | tgcaaggcct | | 687 | gtaacgaacg |
| | 432 | tgcggctcaa | | 560 | gaagacagaa | | 688 | ctcgtaggtc |
| 7 | 433 | taaggctaga | 23 | 561 | acagactcat | 39 | 689 | ctggattact |
| | 434 | gaggagataa | | 562 | gagttgaggc | | 690 | gtatgttcct |
| | 435 | cgttagcact | | 563 | gttctagacg | | 691 | tgttcgcgac |
| | 436 | aggctaggat | | 564 | agttcaggcg | | 692 | cacggaattg |
| | 437 | atctcactgg | | 565 | cagacgatga | | 693 | agcacaatgc |
| | 438 | tccagttctc | | 566 | tccaacctat | | 694 | acacaccgga |
| | 439 | gtaaccgctc | | 567 | ttacgttctc | | 695 | tatctcgcta |
| | 440 | cctcttagcg | | 568 | cgcggtcata | | 696 | gcgatggaag |
| 8 | 441 | actaaggctg | 24 | 569 | aatccttccg | 40 | 697 | taactcgtgg |
| | 442 | gaggagtatg | | 570 | gaggattgaa | | 698 | acaaccagta |
| | 443 | tagtcaacgc | | 571 | atcggagtgc | | 699 | agcgtacgtc |
| | 444 | ccatcaagga | | 572 | gcgttaagtg | | 700 | tcgactctgt |
| | 445 | agcctctgct | | 573 | cctcggcaat | | 701 | ctgtatgcca |
| | 446 | ttcgttcaca | | 574 | ttaacggctt | | 702 | gacgagacct |
| | 447 | gtaagtgtac | | 575 | tgattccaca | | 703 | cgttggtaac |
| | 448 | cgtcgcctat | | 576 | cgcaacatgc | | 704 | gttcgataag |
| 9 | 449 | tgtgaaggag | 25 | 577 | tcaatgaggt | 41 | 705 | caagcgcgat |
| | 450 | agaccggttc | | 578 | gtccaagctg | | 706 | gtgtgagtgc |
| | 451 | ctcgcctaac | | 579 | gcgcactaag | | 707 | gcagcataat |
| | 452 | ccgctgtcta | | 580 | cggtgagtga | | 708 | ttcttgtgca |
| | 453 | gacataacct | | 581 | tgaatccgac | | 709 | tggcacattg |
| | 454 | gagaatagca | | 582 | aatgctcact | | 710 | aacaatacgc |
| | 455 | ttatgtcagg | | 583 | atcggttcca | | 711 | cgtattgctg |
| | 456 | acttgcctgt | | 584 | cattcgattc | | 712 | actcgccaca |
| 10 | 457 | tgctggatct | 26 | 585 | ttatgcctac | 42 | 713 | gctgcttggt |
| | 458 | agtagtgttg | | 586 | atcctccgac | | 714 | ttgtggatac |
| | 459 | ctccaatcct | | 587 | cgcagatata | | 715 | accaaggcga |
| | 460 | cctatgtgta | | 588 | acaacatgtg | | 716 | agaagaccta |
| | 461 | taatatccgc | | 589 | tcggatgagg | | 717 | tagctctgtc |
| | 462 | gtgccacaac | | 590 | cgtgcgatca | | 718 | cgacttctat |
| | 463 | gcagtcagga | | 591 | gatcttacgt | | 719 | ctctccaacg |
| | 464 | aaggccgaag | | 592 | gagtaggcct | | 720 | gatgaagacg |
| 11 | 465 | cgtgttagag | 27 | 593 | aagagagaag | 43 | 721 | tccagctcat |
| | 466 | acaggacgat | | 594 | gttctcacgg | | 722 | accaagactc |
| | 467 | cggataacgg | | 595 | aagcggtgaa | | 723 | caactgcgca |
| | 468 | gttagtgcct | | 596 | cgctcagtta | | 724 | agtggatatc |
| | 469 | aagcacgaca | | 597 | ctagacactc | | 725 | gtactaagag |
| | 470 | gtctccttgc | | 598 | gcttattgct | | 726 | gtgtacgtcg |
| | 471 | tcaccgtata | | 599 | tgcgttctgc | | 727 | cggtctctgt |
| | 472 | tactagcttc | | 600 | tcaacgcact | | 728 | tatgctgaga |
| 12 | 473 | accattcacg | 28 | 601 | cttgcttaca | 44 | 729 | aataggtagg |
| | 474 | agtagctagt | | 602 | ctcctcgcaa | | 730 | agcttgcgct |
| | 475 | ttccgaaggt | | 603 | tctcagcctc | | 731 | tggagccgat |
| | 476 | gtaccaacca | | 604 | gacggagtac | | 732 | ccgcatacta |
| | 477 | tatgtgtgtc | | 605 | acgtctatct | | 733 | ttcgatgacg |
| | 478 | cgatcggtac | | 606 | gaatagagtg | | 734 | gatccaatac |
| | 479 | gaggatctag | | 607 | aggatacggt | | 735 | gcattctcgc |
| | 480 | ccgtacgcta | | 608 | tgaagctagg | | 736 | ctagcagtta |
| 13 | 481 | gtcaactcgg | 29 | 609 | aacctcgcac | 45 | 737 | cgttgacgct |
| | 482 | tggaagcaca | | 610 | gatccggact | | 738 | agtatatgcg |
| | 483 | tcgttgtagc | | 611 | gtgtctacag | | 739 | acctccgcta |
| | 484 | agccgaagtt | | 612 | acggagaatc | | 740 | caacacgaat |
| | 485 | gatgcaacct | | 613 | tgttacctca | | 741 | ttgagtaagg |
| | 486 | acttccgttc | | 614 | tcaatacgtg | | 742 | gaagctctta |
| | 487 | caagttggaa | | 615 | ctaagttggt | | 743 | tccgagatgc |
| | 488 | ctacgtctag | | 616 | cgcggattga | | 744 | gtgctgtcac |
| 14 | 489 | tgtcgttaag | 30 | 617 | acctcacata | 46 | 745 | agcttccagc |
| | 490 | gtctcaatga | | 618 | cggactgtct | | 746 | tgcctatcgc |
| | 491 | gatcaagcca | | 619 | cgtggcagaa | | 747 | caatctcgcg |
| | 492 | aactccgatc | | 620 | attcgaatgc | | 748 | cttcctacaa |
| | 493 | ctaagtcttc | | 621 | taggttcaag | | 749 | acgaggtact |
| | 494 | tgagagcgct | | 622 | gtccactctc | | 750 | gatgaaggat |
| | 495 | acgatctcgg | | 623 | gaatagtgct | | 751 | tcgagcgtta |
| | 496 | ccggtgagat | | 624 | tcaatggcgg | | 752 | gtagagattg |
| 15 | 497 | catctcatga | 31 | 625 | aagcatcctg | 47 | 753 | acggctagag |
| | 498 | ctgtgactcg | | 626 | gtggtgttca | | 754 | gctatagctt |
| | 499 | agaccttgga | | 627 | gtacaacgtt | | 755 | tgcgtcatgg |
| | 500 | actgtcgacg | | 628 | cctgcagcat | | 756 | ttccaacatc |
| | 501 | gtgaagacac | | 629 | tctatcgtac | | 757 | cggtgttgga |
| | 502 | tactgtgcat | | 630 | cactcgtagc | | 758 | gattgcgcct |
| | 503 | gcagagcgtt | | 631 | tgcagcagca | | 759 | ataacgctca |
| | 504 | tgcacatatc | | 632 | agatgtaagg | | 760 | caacagtaac |
| 16 | 505 | cctgtgtaac | 32 | 633 | gtgtaaccgc | 48 | 761 | actcgaggag |
| | 506 | aacgatgcca | | 634 | catcggaaga | | 762 | gccggtaagt |
| | 507 | tcgagcatag | | 635 | gaggaattac | | 763 | ctcacactta |
| | 508 | gaattcctgt | | 636 | tgttcgctct | | 764 | agacagtggc |
| | 509 | agaacgtccg | | 637 | accgtctata | | 765 | ctgatgtcct |
| | 510 | cgccaaggta | | 638 | tgcctcagag | | 766 | gagttcacaa |
| | 511 | ttgtgaaggc | | 639 | acaagtgccg | | 767 | tgttctcttc |
| | 512 | gttcctcatt | | 640 | ctaactggtt | | 768 | taacacgacg |

The splitting rate of sequencing data in the 4-balanced tags groups will be higher, because the sequencing machine reads the bases with the balanced composition more accurately, and the unbalanced bases will cause reading errors and reduce the splitting rate of the sequencing data. When 12 samples are pooled in equal proportions, the 4-balanced tags and 8-balanced tags were both used to construct libraries for sequencing. From the results as shown in FIG. 10, for the 4-balanced tags, the 12 samples have almost the same sequencing data splitting. For the 8-balanced tags, some samples of the 12 samples have significantly reduced data splitting.

Based on the above research results, the inventors proposed the technical solutions of the invention.

In a typical mode of the invention, a double-end library tags composition is provided. The double-end library tags composition includes a plurality of 5′ end library tags and a plurality of 3′ end library tags. The lengths of the 5′ end library tags are all the same, the lengths of the 3′ end library tags are all the same and the occurrences of each base at the same position in the double-end library tags composition are also the same.

In the double-end library tags composition provided by the invention, by controlling the lengths of the 5′ end library tags are all the same, the lengths of the 3′ end library tags are all the same, and occurrences of each base at the same position are all the same, multiple libraries with good- base balanced double-end tags can be obtained. When the multiple libraries are pooled for sequencing, the double-end tags sequence can be read more accurately, and the sequencing data can be split more effectively.

In order to further improve the base balance and reading accuracy of the library tags, in a preferred embodiment, the lengths of the 5′ end library tags are the same with the lengths of the 3′ end library tags, preferably is any fixed length between 6-10 bp. The lengths of the library tags at both ends are the same, so that when the samples are split, the same number of bases in the library tags at both ends participates in determining the source of the sample, so the probability of support provided by the libraries from both ends is the same. It can avoid that one end of the library tag is longer and the reference probability of support is higher, and the other end of the library tag is shorter, and the reference probability of support is lower, which leads to the result that is more biased to rely on tags on one end.

Preferably, in the double-end library tags composition, there are at least 3 base differences between any two library tags, and the number of continuous same bases in any library tag does not exceed 3, preferably, GC contents in all library tags are all 40-60%. When library tags meet the above base optimization principles and are used in combination, the base balance is better, the reading results are accurate, and the data splitting rate is also higher.

Preferably, the double-end library tags composition includes a composition of 4-balanced double-end library tags, or a composition of 8-balanced double-end library tags, the combination of 4-balanced double-end library tags includes 4n 5′ end library tags and 4n 3′ end library tags. The combination of 8-balanced double-end library tags includes 8n 5′ end library tags and 8n 3′ end library tags, n is an integer greater than or equal to 1.

In a preferred embodiment, in the composition of 4-balanced double-end library tags, 5′ end library tags are selected from any one of the 96 groups shown in Table 1, and the 3′ end library tags are selected from any one of the 96 groups shown in Table 1 that is different from the 5′ end library tag group.

In a preferred embodiment, in the composition of 8-balanced double-end library tags, 5′ end library tags are selected from any one of the 48 groups shown in Table 2, and the 3′ end library tags are selected from any one of the 48 groups shown in Table 2 that is different from the 5′ end library tag group.

In the second typical mode of the invention, a composition of amplification primers with double-end library tags based on MGI sequencing platform is provided, and the composition of amplification primers includes a plurality of amplification primer pairs with double-end library tags, each amplification primer pair includes a 5′ end library tag and a 3′ end library tag, and the lengths of the 5′ end library tags are all the same and the lengths of the 3′ end library tags of the amplification primer pairs are all the same, and the occurrences of each base at the same position are also all the same.

By controlling the lengths of 5′ end library tags are all the same and the lengths of the 3′ end library tags of the plurality of amplification primer pairs are all the same, and the occurrences of each base at the same position are also all the same, when the double-end tags in the composition of amplification primers are used to label multiple pooled samples for sequencing, the reading of the tag bases is balanced, the results are more accurate, and the samples data split according to the tags are also more accurate, which improves the splitting rate of the sequencing data.

Based on the same lengths of the 5′ end library tags and the same lengths of the 3′ end library tags of the above pooled samples, in order to further improve the base balance and reading accuracy of the library tags, in a preferred embodiment, the lengths of the 5′ end library tags and the lengths of the 3′ end library tags of the plurality of amplification primer pairs are the same. The lengths of the library tags at both ends of each pair of amplification primers are the same, so that when the samples are split, the same number of bases in the library tags at both ends participates in determining the source of the sample, and the probability of support provided by the libraries at both ends is the same. It can avoid that one end of the library tag is longer and the reference probability of support is higher, and the other end of the library tag is shorter, and the reference probability of support is lower, which leads to the result that is more biased to rely on tags on one end.

More preferably, the lengths of 5′ end library tags and the 3′ end library tags are both any fixed length between 6-10 bp, further the preferred length is 10 bp, which has greater discrimination and more beneficial effects than other lengths such as 6bp or 8 bp.

In order to provide more balanced library tags, in a preferred embodiment, in the composition of amplification primers, there are at least 3 base differences between any two library tags, and the number of the continuous same base in any one of the library tags does not exceed 3, and the GC contents of the library tags are all 40-60%. When library tags meet the above base optimization principle and are used in combination, the balance of base reading is better, the result is more accurate, and the splitting rate of the sequencing data is also higher.

In a preferred embodiment, the mentioned composition of amplification primers includes a combination of 4n 4-balanced tags amplification primer pairs, or a combination of 8n 8-balanced tags amplification primer pairs, where n is an integer greater than or equal to 1. More preferably, in the 4n 4-balanced tags amplification primer pairs, the 5′ end library tags are selected from any one or more of the 96 groups shown in Table 1, and the 3′ end library tags are selected from any one or more of the 96 groups different from the 5′-end library tags shown in Table 1. The number of groups here is determined according to the actual needs. The combinations of 96 groups of tag sequences in Table 1 makes higher reading accuracy, so sequencing data splitting is more accurate, and the splitting rate is also higher.

In another preferred embodiment, in the 8n amplification primer pairs with 8-balanced tags, the 5′ end library tags are selected from any one or more of the 48 groups shown in Table 2, and the 3′ end library tags are selected from any one or more of the 48 groups shown in Table 2 that are different from the 5′ end of the library tag groups.

In the above composition of amplification primers, each amplification primer pair further includes a 5′ end universal amplification sequence and a 3′ end universal amplification sequence, and the 5′-end universal amplification sequence includes the universal downstream sequence of the 5′ end library tags and the universal upstream sequence of the 5′ end library tags, the 3′ end universal amplification sequence includes the universal downstream sequence of the 3′ end library tags and the universal upstream sequence of the 3′ end library tags. The specific sequence of the universal amplification sequence in each amplification primer pair is determined according to the existing universal sequences of MGI sequencing platform. The combination of amplification primers formed by the amplification primer pairs containing the above library tags can improve the reading accuracy of the library tags when the samples are pooled and sequenced on the machine, thereby improving the accuracy of the sequencing data of each sample.

As mentioned above, the library construction can adopt a relatively short bubble adapter (that is the number of unpaired bases in the middle region is 30±5 bp), or a relatively long bubble adapter (the number of unpaired bases in the middle region is 45±5 bp). Correspondingly, the universal sequence in the amplification primer pair here can also be adjusted to a longer or shorter universal amplification sequence according to the length of the bubble adapter.

In a preferred embodiment, corresponding to the use of a shorter bubble adapter, the universal upstream sequence of the 5′ end library tag is SEQ ID NO: 793, and the universal downstream sequence of the 5′ end library tag is SEQ. ID NO: 794; the universal upstream sequence of the 3′ end library tag is SEQ ID NO: 795, and the universal downstream sequence of the 3′ end library tag is SEQ ID NO: 796.

In another preferred embodiment, corresponding to the use of a longer bubble adapter, the universal upstream sequence of the 5′ end library tag is SEQ ID NO: 793, and the universal downstream sequence of the 5′ end library tag is SEQ. ID NO: 797; the universal upstream sequence of the 3′ end library tag is SEQ ID NO: 795, and the universal downstream sequence of the 3′ end library tag is SEQ ID NO: 798.

In the third mode of the invention, a library construct kit based on MGI sequencing platform is also provided, the kit includes any one the composition of amplification primers mentioned above. The double-end library tags in the amplification primers have the base balance, so the tag sequences of each sample after the sequencing can be accurately read, and the data split accuracy of the pooled samples are improved.

In order to further improve the convenience of the library construction, the kit may further includes a bubble adapter of the MGI sequencing platform, the bubble adapter includes a first adapter sequence and a second adapter sequence, and the first adapter sequence is SEQ ID NO: 769, the second adapter sequence is SEQ ID NO: 770, or the first adapter sequence is SEQ ID NO: 773, the second adapter sequence is SEQ ID NO: 774. Compared to a relatively longer bubble adapter, the shorter bubble adapter can not only improve the stability of the ligation and have higher ligation efficiency, but also is more compatible in the subsequent PCR amplification procedures after the adapter ligation.

In the fourth embodiment of the invention, a method of constructing a sequencing library applying any of the above kits based on MGI sequencing platform is provided. When the libraries constructed as the above kits are sequenced on the machine, the balance of the library tags is better, and the reading accuracy data splitting rate are higher.

In the fifth embodiment of the invention, a sequencing library is also provided. The sequencing library includes any of the composition of amplification primers, or is constructed through any of the above methods. The balance of the library tags in the sequencing library is better, and the read accuracy of the library tags after sequencing is higher, and the data splitting rate is higher.

The advantages of the invention will be further described below in the embodiment. It should be noted that the following examples uses NadPrep™ DNA library prep kit to construct the libraries.

Item No.: 1002212 NadPrep® Plasma Free DNA double-end tag library prep kit (for MGI).

Item No.: 1003811 User's Guide V1. 0 (Nanodigmbio, Nanjing).

The process is briefly described as follows.

DNA Sample Fragment—End Repair and A-Tailing—Ligation—Fragment Selection—PCR Amplification—Library Purification, Quantitative and Quality Control—Sequencing or targeting Sequencing on MGI platform.

It will also be noted that the following examples are merely exemplary, and do not limit the method of the invention to be the following methods.

### EXAMPLE 1 LIBRARY PREP SOLUTION 1 and SOLUTION 2

Steps: Refer to NadPrep™ DNA library prep kit (for MGI) (201909Version2.0) The differences lie in the bubble adapter sequence and the amplification primer sequence.

### (1)Solution1:

Bubble adapter sequence:
SEQ ID NO:769 (adapter 1) and SEQ ID NO:770 (adapter 2):

SEQ ID NO: 769:
(31 bp)/phos/agtcggaggccaagcggtcttaggaagacaa;
SEQ ID NO: 770(40 bp):
ttgtcttcctaacaggaacgacatggctacgatcogact*t.

SEQ ID NO:771 (amplification primer 1) [symbol] SEQ ID NO:772 (amplification primer 2):
SEQ ID NO:771: (64bp)
/phos/ctctcagtacgtcagcagttnnnnnnnnnncaactccttggctcacagaacgacatggctacga, wherein the sequence before nnnnnnnnnn (/phos/ctctcagtacgtcagcagtt) is SEQ ID NO: 793, the sequence after nnnnnnnnnn (caactccttggctcacagaacgacatggctacga) is SEQ ID NO: 794, (gacatagctacga is the prolonged part compared to the solution 2).
SEQ ID NO:772: (52bp)
gcatggcgaccttatcagnnnnnnnnnnttgtcttcctaagaccgcttggcc, wherein the sequence before nnnnnnnnnn (gcatggcgaccttatcag) is SEQ ID NO:795, the sequence after nnnnnnnnnn (ttgtcttcctaagaccgcttggcc) is SEQ ID NO:796 (cc is the prolonged part compared to the solution 2).

### Characteristics of Solution 1:

1. The complementary portion of the adapter is 7+13 bp (belong to the region of 20±2 bp), the vesicle structure region is 20±12 bp (belong to the region of 30±5 bp);
2. The amplification primer is a little longer.

### Advantages:

1. The vesicle structure is shorter, so the annealed structure is stable.
2. The amplification primer is compatible to single-end amplification primers and single-end tags (see the CN application NO. 201910229527.4).

### (2)Solution 2:

Adapter sequence
SEQ ID NO: 773 (adapter 1) [symbol] SEQ ID NO: 774 (adapter 2).

SEQ ID NO: 773(35 bp):
/phos/agtcggaggccaagcggtcttaggaagacaatcag.
SEQ ID NO: 774(59 bp):
ctgattgtcttcctaagcaactccttggctcacagaacgacatggcta
cgatccgactt.

SEQ ID NO:775 (amplification primer 1) [symbol] SEQ ID NO:776 (amplification primer 2).
SEQ ID NO:775: (51bp)
/phos/ctctcagtacgtcagcagttnnnnnnnnnncaactccttggctcacagaac; wherein the sequence before nnnnnnnnnn (/phos/CTCtcagtacgtcagcagtt) is SEQ ID NO:793, the sequence after nnnnnnnnnn (caactccttggotcacagaac) is SEQ ID NO:797.
SEQ ID NO:776: (50bp)
gcatggcgaccttatcagnnnnnnnnnnttgtcttcctaagaccgottgg, wherein the sequence before nnnnnnnnnn (gcatggcgaccttatcag) is SEQ ID NO:795, the sequence after nnnnnnnnnn (ttgtcttcctaagaccgcttgg) is SEQ ID NO:798.

### Characteristics of Solution 2:

1.The complementary portion of the adapter is 7±17 bp (belong to the region of 25±2 bp), the vesicle structure is 34±12 bp (belong to the region of 45±5 bp);
2. The amplification primer is shorter.
   Disadvantages Compared with the Solution 1:
1. The vesicle structure is longer, so the annealed structure is relatively unstable.
2. The amplification primer is not compatible to other solutions (amplification primer is shorter, and there is no repeat sequence with the vesicle structure).

The results of the adapter structures and amplification primers of the solution 1 and solution 2 are shown in FIG. 4. The libraries with double-end tags for MGI sequencing can both be obtained. 25 ng and 100 ng DNA are input for library construction in experiment process. The information is shown in the table below.

TABLE 3
Library yields from solution 1 and solution 2

| Solution | DNA Input | | PCR cycles | Library yield |
|---|---|---|---|---|
| 1 | 25 | ng | 7 | 1222 ng |
| | 100 | ng | 5 | 1367 ng |
| 2 | 25 | ng | 7 | 1176 ng |
| | 100 | ng | 5 | 1159 ng |

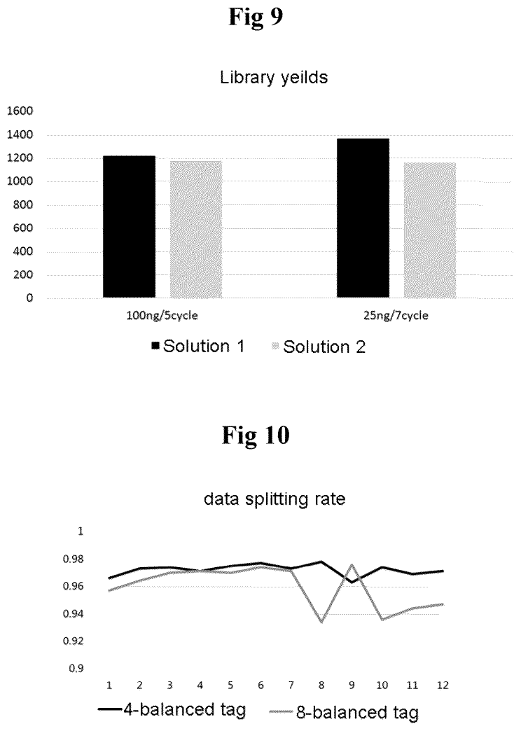

The libraries with double-end tags for MGI can both be obtained from solution 1 and solution 2, and the library yields are similar, as shown in FIG. 9. But the solution 2 is not compatible to the single-end amplification primers and adapters with single-end tags.

### EXAMPLE 2 COMPARISON OF DATA SPLITTING IN 12 POOLED SAMPLES BETWEEN 4-BALANCED TAGS AND 8-BALANCED TAGS

The solution using double-end tags can effectively solve the crosstalk problems between samples (also called the tag jumping). But only when both ends of the tags are correct, the sequencing data can be effectively split. So the double-end tags balance requirements are more stringent than the single-end tags. The present invention optimizes two set of solutions with 4-balanced tags and 8-balanced tags. This example adopted both 4-balanced tags and 8-balanced tags, and pooled 12 libraries for sequencing to detect splitting rate of each sample in two set of solutions. The experimental steps and information are as follows:

Steps: Refer to NadPrep™ DNA library prep kit (for MGI) (201909Version2.0) instructions. The only difference lies in that the adapter with single-end tags was changed into the adapter with double-end tags.

The 4-balanced tags sequence used in the experiment is shown in Table 4, adjacent 4 tags are a group of balance, and each group is distinguished with bold or non-thickened fonts. The tag 1 is a forward arrangement of 384 tag sequences, and the tag 2 is a reverse arrangement of 384 tag sequences. The primer1 with tag 1 and the primer 2 with the tag 384 constitute the combination of the first group of double-end tag primers. The primer1 with tag 2 and the primer 2 with the tag 383 constitute the combination of the second group of double-end tag primers. Totally there will be 384 combinations.

8-balanced tags arrangements and 4-balanced tags arrangements are the same. The only difference is 8 tags in a group, as shown in Table 5. When 12 library tags are put together, the first 8 is balanced, the last 4 is unbalanced. For the 4-balanced tags combination, the 12 library tags are exactly balanced.

TABLE 4
The 12 4-balanced tags combinations

| Combination | | | | |
|---|---|---|---|---|
| No. | Tag 1 No. | Tag 1 SEQ | Tag 2 No. | Tag 2 SEQ |
| XDI001 | 1(SEQ ID NO: 1) | tcacattgct | 384(SEQ ID NO: 384) | gatagtaacg |
| XDI002 | 2(SEQ ID NO: 2) | aatggcgctc | 383(SEQ ID NO: 383) | tgagtggcta |
| XDI003 | 3(SEQ ID NO: 3) | gtctcaatga | 382(SEQ ID NO: 382) | ccgtcattac |
| XDI004 | 4(SEQ ID NO: 4) | cggatgcaag | 381(SEQ ID NO: 381) | atccaccggt |
| XDI005 | 5(SEQ ID NO: 5) | tcgcttaagc | 380(SEQ ID NO: 380) | gcaactgtga |
| XDI006 | 6(SEQ ID NO: 6) | cgaggcttag | 379(SEQ ID NO: 379) | atccaccacc |
| XDI007 | 7(SEQ ID NO: 7) | gtctaaggct | 378(SEQ ID NO: 378) | cgtgtgacat |
| XDI008 | 8(SEQ ID NO: 8) | aatacgccta | 377(SEQ ID NO: 377) | tagtgatgtg |
| XDI009 | 9(SEQ ID NO: 9) | aagcctattg | 376(SEQ ID NO: 376) | gcttgttcag |
| XDI010 | 10(SEQ ID NO: 10) | cgctactgca | 375(SEQ ID NO: 375) | aacaagcact |
| XDI011 | 11(SEQ ID NO: 11) | tcaagagcat | 374(SEQ ID NO: 374) | ttgccagtga |
| XDI012 | 12(SEQ ID NO: 12) | gttgtgcagc | 373(SEQ ID NO: 373) | cgagtcagtc |

TABLE 5
The 12 4-balanced tags combinations

| Combination | | | | |
|---|---|---|---|---|
| No. | Tag 1 No. | Tag 1 SEQ | Tag 2 No. | Tag 2 SEQ |
| MDI001 | 1(SEQ ID NO: 385) | cgtcgatgac | 384(SEQ ID NO: 768) | taacacgacg |
| MDI002 | 2(SEQ ID NO: 386) | atataaggcg | 383(SEQ ID NO: 767) | tgttctcttc |
| MDI003 | 3(SEQ ID NO: 387) | gatcgtgctc | 382(SEQ ID NO: 766) | gagttcacaa |
| MDI004 | 4(SEQ ID NO: 388) | cagtcttcgg | 381(SEQ ID NO: 765) | ctgatgtcct |
| MDI005 | 5(SEQ ID NO: 389) | agaacgatct | 380(SEQ ID NO: 764) | agacagtggc |
| MDI006 | 6(SEQ ID NO: 390) | ttggtgcatt | 379(SEQ ID NO: 763) | ctcacactta |
| MDI007 | 7(SEQ ID NO: 391) | gccgtcataa | 378(SEQ ID NO: 762) | gccggtaagt |
| MDI008 | 8(SEQ ID NO: 392) | tccaaccaga | 377(SEQ ID NO: 761) | actggaggag |
| MDI009 | 9(SEQ ID NO: 393) | gatagcaaga | 376(SEQ ID NO: 760) | caacagtaac |
| MDI010 | 10(SEQ ID NO: 394) | accgtgcttc | 375(SEQ ID NO: 759) | ataacgctca |
| MDI011 | 11(SEQ ID NO: 395) | gcagatgtaa | 374(SEQ ID NO: 758) | gattgcgcct |
| MDI012 | 12(SEQ ID NO: 396) | tgttggagcg | 373(SEQ ID NO: 757) | cggtgttgga |

For the human genomic DNA standard, libraries are constructed with 12 combinations of double-end 4-balanced tags and 8-balanced tags. The double-end 4-balanced tags sequences are shown in Table 4, and the double-end 8-balanced tags sequences are shown in Table 5. The 4-balanced libraries and 8-balanced libraries are sequenced and analyzed on MGI sequencing platform.

The two groups of libraries were splitting for two rounds, in the first round, the maximum fault tolerance (will split the sequencing error) was used for splitting, and in the second round, only one fault tolerance per tag was allowed for splitting. The results of data splitting were shown in FIG. 10, the data splitting rate of the 12 libraries with 4-balanced tags is more stable, and the data splitting rate of the 12 libraries with 8-balanced tags is not stable. The results show that the balanced double-end tags are more conducive to the effective data splitting of the MGI sequencer, herein the design of 8-balanced tags improves the data effective splitting rate to some extent, and the design of 4-balanced tags is better.

### EXAMPLE 3

To ensure the performance difference between 48 groups of 8-balanced tags combinations provided by the present invention and the 12 groups of 8-balanced tags combinations provided by MGI manufacturing, the compatibility was considered when they were designed. There are 3 bases difference in any two sequences between 48 groups of 8-balanced tags combinations and 12 groups of 8-balanced tags combinations provided by MGI manufacturing.

In addition, there are other major distinguishes as follows:
1. The base composition of the tag sequence in the present invention is more equalized, and the GC content is 40% -60%, but the GC content of tags from MGI manufacturing is from 20% to 80%.
2. The tag sequence of the present invention performs a matching property of the adapter sequence of the solution 1 to ensure amplified libraries to be evenly produced. But some sequences from MGI manufacturing are not satisfied with the balanced requirement on library amplification efficiency.

In order to further verify the performance difference in amplification balance, a group of 8-balanced tags combinations MDI001-MDI008 of the invention and a group of 8-balanced tags combinations MGI001-MGI008 from MGI manufacturing (shown in Table 6) were selected to construct libraries: 100 ng of DNA as input, PCR amplification for 5 cycles to detect the library yields, and the results were shown in Table 7.

As shown in Table 7. the library yields from the invention are equal, while one library yield from MGI manufacturing is less than half of the normal value, which indicates that the optimized tag sequences of the present invention has better balance. Further, amplification efficiency is more stable. At the same time, due to the high throughput of the MGI sequencer, the two groups of 384 tags in the present invention are better than the 120 tags from MGI manufacturing to meet the throughput demand for pooled sequencing.

TABLE 6
8 combinations of 8-balanced tags from MGI manufacturing

| Combination | | | | |
|---|---|---|---|---|
| No. | Tag 1 No. | Tag 1 SEQ | Tag 2 No. | Tag 2 SEQ |
| MGI001 | 1(SEQ ID NO: 777) | atgcatctaa | 120(SEQ ID NO: 785) | tagaggacaa |
| MGI002 | 2(SEQ ID NO: 778) | agctctggac | 119(SEQ ID NO: 786) | cctagcgaat |
| MGI003 | 3(SEQ ID NO: 779) | ctatcacgtg | 115(SEQ ID NO: 787) | gtagtcatcg |
| MGI004 | 4(SEQ ID NO: 780) | ggactagtgg | 117(SEQ ID NO: 788) | gctgagctgt |
| MGI005 | 5(SEQ ID NO: 781) | gccaagtcca | 116(SEQ ID NO: 789) | aacctagata |
| MGI006 | 6(SEQ ID NO: 782) | cctgtcaagc | 115(SEQ ID NO: 790) | ttgccatctc |
| MGI007 | 7(SEQ ID NO: 783) | tagaggtctt | 114(SEQ ID NO: 791) | agatcttgcg |
| MGI008 | 8(SEQ ID NO: 784) | tatggcaact | 113(SEQ ID NO: 792) | cgctatcggc |

TABLE 7

| Library | Library | Library | Library |
|---|---|---|---|
| No. | Yield | No. | Yield |
| MGI001 | 1328 | MDI001 | 1386 |
| MGI002 | 1251 | MDI002 | 1255 |
| MGI003 | 1196 | MDI003 | 1229 |
| MGI004 | 1267 | MDI004 | 1311 |
| MGI005 | 667 | MDI005 | 1307 |
| MGI006 | 1345 | MDI006 | 1238 |
| MGI007 | 1257 | MDI007 | 1233 |
| MGI008 | 1344 | MDI008 | 1274 |

From the above embodiments, in the present invention double-end library tags are introduced on MGI sequencing platform to solve the samples crosstalk problems caused by the synthesis, the experimental process, and the sequencing process, which will make the detection results more accurate. Furthermore, the inventors found that through test and optimization, when the middle structure of the bubble adapter is 30±5 bp, the paired base is 20±2 bp, the annealing of the vesicle adaptors is most stable. Meanwhile, the amplification primer is an extended amplification primer, which can be compatible with the amplicons with single-end tags and adapters with molecular single-end tags. The bubble adapters with such a compositional structure are used together with the extended amplification primers in the library construction, which can be compatible with the existing single-end tags solution of MGI platform, and is convenient for the MGI sequencing application.

Based on the above, in order to obtain a better data splitting, the present invention optimized 384 combinations of 4-balanced tags and 8-balanced tags sequences, respectively, which provides optimal solution for high-throughput sequencing and sequencing data splitting for MGI platform.

The above description is only the preferred embodiment of the present invention, and is not intended to limit the present invention, and various modifications and changes can be made to the present invention for those skilled in the art. Any modifications, equivalent substitutions, improvements, etc. made within the spirit and scopes of the present invention are intended to be included within the protection scopes of the present invention.

What is claimed is:

1-4. (canceled)

5. A composition of amplification primers with double-end library tags based on MGI sequencing platform, comprising:
a plurality of amplification primer pairs with double-end library tags, each amplification primer pair comprises a 5′ end library tag and a 3′ end library tag,
wherein the lengths of multiple 5′ end library tags of the amplification primer pairs are all the same, and the lengths of multiple 3′ end library tags of the amplification primer pairs are all the same, and the occurrences of each base at the same position are also all the same.

6. The composition as claimed in claim 5, wherein the lengths of multiple 5′ end library tags of the amplification primer pairs are all the same with the lengths of multiple 3′ end library tags of the amplification primer pairs;
preferably, the lengths of the multiple 5′ end library tags and the lengths of the multiple 3′ end library tags are any fixed lengths between 6˜10 bp;
preferably, in the composition, there are at least 3 base differences between any two library tags, and the number of continuous same bases in any library tag does not exceed 3;
preferably, GC contents in all library tags are all 40-60%;
preferably, the composition comprises a combination of 4n 4-balanced amplification primer pairs, or a combination of 8n 8-balanced amplification primer pairs, wherein n is an integer greater than or equal to 1.

7. The composition as claimed in claim 6, wherein in the combination of 4n 4-balanced amplification primer pairs, the 5′ end library tags are selected from any one or more of the 96 groups shown in Table 1, and the 3′ end library tags are selected from any one or more of the 96 groups shown in Table 1 that are different from the 5′-end library tags;
preferably, wherein in the combination of 8n 8-balanced amplification primer pairs, the 5′ end library tags are selected from any one or more of the 48 groups shown in Table 2, and the 3′ end library tags are selected from any one or more of the 48 groups shown in Table 2 that are different from the 5′-end library tags.

8. The composition as claimed in, wherein each amplification primer pair further comprises a 5′ end universal amplification sequence and a 3′ end universal amplification sequence, the 5′ end universal amplification sequence comprises an universal upstream sequence of the 5′ end library tag and an universal downstream sequence of the 5′ end library tag, and the 3′ end universal amplification sequence comprises an universal upstream sequence of the 3′ end library tag and an universal downstream sequence of the 3′ end library tag;
preferably, the universal upstream sequence of the 5′ end library tag is SEQ ID NO: 793, and the universal downstream sequence of the 5′ end library tags is SEQ ID NO: 794; the universal upstream sequence of the 3′ end library tag is SEQ ID NO: 795, and the universal downstream sequence of the 3′ end library tag is SEQ ID NO: 796; or
the universal upstream sequence of the 5′ end library tag is SEQ ID NO: 793, and the universal downstream sequence of the 5′ end library tag is SEQ ID NO: 797; the universal upstream sequence of the 3′ end library tag is SEQ ID NO: 795, and the universal downstream sequence of the 3′ end library tag is SEQ ID NO: 798.

9-10. (canceled)

11. A method for constructing a sequencing library based on MGI sequencing platform, comprising applying the composition of amplification primers as claimed in claim 5 to construct.

12. A sequencing library, comprising the combination of amplification primers as claimed in claims 5.

13. The method as claimed in claim 11, wherein the method comprises the following steps:
1. DNA sample fragmentation, 2) end repair and A-tailing, 3) adapter ligation, 4) fragment selection and 5) PCR amplification, respectively,
wherein in the step 3) of adapter ligation, the adapter is bubble adapters, wherein the bubble adapters comprise a first adapter sequence and a second adapter sequence, the first adapter sequence is SEQ ID NO: 769, and the second adapter sequence is SEQ ID NO: 770, or
the first adapter sequence is SEQ ID NO: 773, and the second adapter sequence is SEQ ID NO: 774.

14. The method as claimed in claim 13, wherein,
when the first adapter sequence is SEQ ID NO: 769, and the second adapter sequence is SEQ ID NO: 770, in the step of 5) PCR amplification, applying the composition of amplification primers shown in SEQ ID NO:771 and SEQ ID NO:772 to perform the PCR amplification;
when the first adapter sequence is SEQ ID NO: 773, and the second adapter sequence is SEQ ID NO: 774, in the step of 5) PCR amplification, applying the composition of amplification primers shown in SEQ ID NO: 775 and SEQ ID NO:776 to perform the PCR amplification.

15. The method as claimed in claim 14, wherein the composition of amplification primers includes a plurality of amplification primer pairs with double-end library tags, each amplification primer pair comprises a 5′ end library tag and a 3′ end library tag, and the lengths of multiple 5′ end library tags of the amplification primer pairs are all the same, and the lengths of multiple 3′ end library tags of the amplification primer pairs are all the same, and the occurrences of each base at the same position are also all the same.

16. The method as claimed in claim 13, wherein the lengths of multiple 5′ end library tags of the amplification primer pairs are all the same with the lengths of multiple 3′ end library tags of the amplification primer pairs;
preferably, the lengths of the multiple 5′ end library tags and the lengths of the multiple 3′ end library tags are any fixed lengths between 6˜10 bp;
preferably, in the composition, there are at least 3 base differences between any two library tags, and the number of continuous same bases in any library tag does not exceed 3;
preferably, GC contents in all library tags are all 40-60%.

17. The method as claimed in claim 16, wherein the composition comprises a combination of 4n 4-balanced amplification primer pairs, or a combination of 8n 8-balanced amplification primer pairs, wherein n is an integer greater than or equal to 1.

18. The method as claimed in claim 17, wherein in the combination of 4n 4-balanced amplification primer pairs, the 5′ end library tags are selected from any one or more of the 96 groups shown in Table 1, and the 3′ end library tags are selected from any one or more of the 96 groups shown in Table 1 that are different from the 5′-end library tags.

19. The method as claimed in claim 17, wherein in the combination of 8n 8-balanced amplification primer pairs, the 5′ end library tags are selected from any one or more of the 48 groups shown in Table 2, and the 3′ end library tags are selected from any one or more of the 48 groups shown in Table 2 that are different from the 5′-end library tags.
